(19) United States
(12) Patent Application Publication
(10) Pub. No.: US 2020/0085985 A1
(43) Pub. Date: Mar. 19, 2020

(54) MULTIPLE CHAMBER DECONTAMINATION DEVICE WITH A STERILIZING SUBSTANCE

(71) Applicant: Mikael Naygauz, Forrest Hills, NY (US)

(72) Inventor: Mikael Naygauz, Forrest Hills, NY (US)

(21) Appl. No.: 16/168,759

(22) Filed: Oct. 23, 2018

Related U.S. Application Data
(60) Provisional application No. 62/733,011, filed on Sep. 18, 2018.

(51) Int. Cl. A61L 2/26; A61L 2/18; A61L 2/00

(52) U.S. Cl. CPC A61L 2/26; A61L 2/0088; A61L 2/0017; A61L 2/18

(57) ABSTRACT

The present invention relates, in some embodiments thereof, to devices and methods for decontaminating a surface of one or more vessels. In some embodiments, the devices of the invention include a first and a second chamber wherein the first chamber configured to accommodate a sterilizing substance and is fluidly connected to the second chamber, the second chamber configured to connect at least one vessel and to receive the sterilizing substance from the first chamber, wherein upon transferring the sterilizing substance to the second chamber, the surface of the at least one vessel is decontaminated.

### CROSS REFERENCE TO RELATED APPLICATIONS

This application claims the benefit of U.S. Provisional Application No. 62/733,011 entitled “Multiple Chamber Decontamination Device with a Sterilizing Substance” filed Sep. 18, 2018. The contents of this application are incorporated by reference in its entirety.

### FIELD OF THE INVENTION

The present invention relates, in some embodiments thereof, to devices and methods for decontaminating a surface of one or more vessels. In some embodiments, the devices of the invention include a first and a second chamber wherein the first chamber configured to accommodate a sterilizing substance and is fluidly connected to the second chamber, the second chamber configured to connect at least one vessel and to receive the sterilizing substance from the first chamber, wherein upon transferring the sterilizing substance to the second chamber, the surface of the at least one vessel is decontaminated.

### BACKGROUND OF THE INVENTION

The medicinal practice routinely involves administration of medical substances, such as, medicaments, fluids, nutrition substances and the alike, to patients. The preparation and/or administration of such medicinal substances typically involves one or more transfers of those substances between vessels (such as, vial, syringes, infusion lines, etc.). Each such act of transferring exposes connection interfaces of the vessels and accordingly the medical substances themselves to contaminants present in ambient air particles (e.g., bacteria, viruses, funguses, spores, pyrogens). In addition, connection interfaces are further prone to contaminations due to physical contact of the interfaces, for example, with nonsterile gloves, or devices.

Such contaminations are a major problem in the healthcare setting since those, once invading within medicinal substances, may pose substantial danger to patients.

Typical connection interfaces of medicaments include rubber bungs/stoppers covered by a cap/seal that can be flicked off/removed prior to usage thereof. Those rubbers are used to allow penetration by a needle attached to a syringe or by other medical connectors. When the cap is flicked off/removed, the rubber bung/stopper is exposed to ambient air and to contaminants presents therein. Accordingly, exposure of connection interfaces to ambient air may involve contamination of the interfaces and consequently contamination of a beneficial substance to be provided to a patient.

Existing systems include U.S. Pat. Nos. D720,067; D717,947; D703,812; D690,418; D639,939; D637,713; U.S. Pat. Nos. 9,790,011; 9,775,777; 9,561,326; 9,493,281; 9,492,353; 9,309,020; 9,173,816; 9,168,203; 9,162,803; 9,039,672; 8,926,583; 8,827,978; 8,790,330; 8,662,985; 8,657,803; 8,622,985; 8,562,583; 8,545,475; 8,523,838; 8,491,563; 8,480,646; 8,449,521; 8,381,776; 8,336,587; 8,328,772; 8,287,513; 8,225,826; 8,075,550; 8,029,747; 7,998,134; 7,975,733; 7,942,860; 7,867,215; 7,744,581; 7,731,678; 7,387,216; 7,306,584; 6,875,203; 6,729,370; 6,715,520; 6,602,239; 6,409,708; 6,343,629; 6,162,199; 6,113,583; 6,063,068; 5,893,397; 5,876,380; 5,832,971; 5,807,374; 5,746,733; 5,569,235; 5,462,535; 5,405,326; 5,292,318; 5,279,582; 4,944,723; 4,932,947; 4,932,937; 4,919,657; 4,915,701; 4,826,489; 4,673,404; 4,564,054; 3,610,241; 3,605,743; 3,587,575; 3,583,399; 3,578,037; 3,556,099; 3,552,387; 3,406,686; 3,380,450; 3,375,825; 3,342,180; 3,330,282; 3,330,281; 3,306,290; 3,255,752; 3,253,592; 3,076,456; 2,972,991; 2,922,419; US20160262982; US20160038373; US20150209568; US20140183196; US20140016570; US20140007973; US20140000754; US20130184672; US20130006200; US20120209238; US20120209218; US20120203194; US20110284561; US20110186177; US20110125128; US20110108158; US20110098647; US20100249745; US20100198182; US20100152669; US20100147402; US20100036319; US20100004602; US20090057258; US20080312634; US20080223484; US20080171981; US20060276759; US20050215976; US20030199847; US20030187420; US20020130100; US20020115981; US20020099354; ES2577377T3; EP2852367B1; EP2666513; EP2155141B1.

In order to overcome this obstacle, the current medical practice involves swabbing the surface of a connection interface with a disinfecting agent, such as 70% isopropyl alcohol, prior to accessing the connection interface. Other methods include i.v. (intravenous) rooms which are used for the sterile preparation of i.v. medications. Such rooms, to keep medicinal preparations as sterile as possible, should be equipped with special instruments including, hoods with air filtrations (e.g., HEPA filters), ventilation systems and air pressure systems. Additionally, those rooms necessitate the medical staff to be properly garmented, use aseptic techniques, and employ quality control and validation processes. These systems are therefore expensive, labor intensive, and require regular maintenance and testing to assure that they are operating effectively.

There is thus a long felt and unmet need for devices and/or methods that afford transfer of medical substances in a sterile manner. There is a need for reliable, user friendly and cost-effective solutions allowing contaminant free engagement of medical devices, for drug preparation and administration processes.

### SUMMARY OF THE INVENTION

It is an object of the present invention, in some embodiments, to provide systems, devices and methods which allow to decontaminate medical vessels in a simple and efficient manner.

It is an object of the present invention, in some embodiments, to provide systems, devices and methods allowing such attributes that are also cost effective.

Objects of the invention are achieved, in some embodiments, by providing systems, devices and methods for transferring medical substances between vessels and administering same to patients in a decontaminated manner.

Objects of the invention are achieved, in some embodiments, by providing systems, devices and methods which afford the transfer of medical substances in an efficient, user-friendly and essentially sterile manner.

Objects of the invention are achieved, in some embodiments, by providing systems, devices and methods for decontaminating medical vessels which abolish the need for the known cumbersome and expensive decontamination techniques.

The present invention provides devices that allow to decontaminate connection surface(s) of medical or pharmaceutical vessels and thereafter permit fluid passageway between the vessels. Due to the first step of decontamination of the vessels, the fluid passageway formed subsequently between the vessels is therefore substantially sterile.

In a first aspect, the present invention provides a device for decontaminating a surface of at least one vessel, the device comprising: a housing comprising a first chamber configured to accommodate a sterilizing/disinfecting substance; and a second chamber, connected to the first chamber and configured to connect the at least one vessel, the second chamber further configured to receive the sterilizing substance from the first chamber and to thereby decontaminate the surface of the at least one vessel.

In one or more embodiments, the second chamber is fluidly connected to the first chamber.

In one or more embodiments, the device further includes an entry aperture disposed between the first chamber and the second chamber, the entry aperture is configured to allow the sterilizing/disinfecting substance to enter from the first chamber to the second chamber.

In one or more embodiments, the entry aperture is sealed by a cover. In one or more embodiments, the entry aperture includes a one-way valve.

In one or more embodiments, the second chamber comprises an exit aperture for allowing removal of the sterilizing/disinfecting substance from the second chamber.

In one or more embodiments, the exit aperture is sealed by a cover. In one or more embodiments, the exit aperture includes a one-way valve.

In one or more embodiments, the device further comprises an entry actuator operable to manipulate opening of the entry aperture.

In one or more embodiments, opening the entry aperture includes opening a valve or a cover disposed along the entry aperture.

In one or more embodiments, the entry actuator is connected to the first chamber from an exterior surface thereof.

In one or more embodiments, the entry actuator is selected from a group consisting of a handle, a tab, and a button.

In one or more embodiments, the device further comprising an exit actuator operable to open the exit aperture, thereby allowing removal of the sterilizing substance from the second chamber.

In one or more embodiments, opening the exit aperture includes opening a valve or a cover disposed along the exit aperture.

In one or more embodiments, the exit actuator is connected to the first chamber from an exterior surface thereof.

In one or more embodiments, the exit actuator is selected from a group consisting of a handle, a tab, and a button.

In one or more embodiments, the second chamber comprises a first connection port configured for coupling to a first vessel, wherein the first connection port provides a connection between the second chamber and the first vessel.

In one or more embodiments, the device further comprises a second connection port configured for coupling to a second vessel, wherein the second connection port provides a connection between the second chamber and the second vessel.

In one or more embodiments, the first and second connection ports are further configured to allow a fluid passageway between the first vessel and the second vessel.

In one or more embodiments, the fluid passageway between the first and second vessels is via the second chamber.

In one or more embodiments, at least one of the first and second connection ports are configured to allow evaporation of the sterilizing substance from the second chamber.

In one or more embodiments, the sterilizing substance is selected from the group consisting of an antibacterial substance (bactericidal substance), an antiviral substance (virucidal substance), an anti-tuberculin substance, and a combination thereof.

In one or more embodiments, the sterilizing substance is selected from the group consisting of ethanol, glutaraldehyde, formaldehyde, hydrogen peroxide, peracetic acid, chlorine, a phenolic compound, guanidinium thiocyanate, sodium hydroxide, isopropanol, (70% or greater concentration) and a combination thereof.

In one or more embodiments, the sterilizing substance is a liquid or a gas. In one or more embodiments, the sterilizing substance is pressurized. In one or more embodiments, the first chamber has a greater pressure than the second chamber. In one or more embodiments, the first chamber has a pressure greater than ambient air pressure.

In one or more embodiments, the device further comprising a sterilizing substance transfer mechanism configured to allow transferring of the sterilizing substance from the first chamber to the second chamber.

In one or more embodiments, the second chamber has an engagement mechanism to connect the at least one vessel to the second chamber.

In one or more embodiments, the engagement mechanism is selected from a thread, a luer, a ratchet teeth mechanism, a retention member, a sliding rail mechanism, a clamping arms mechanism and an adhesive mechanism.

In one or more embodiments, the engagement mechanism positions the surface of the at least one vessel at about same level as an interior wall of the second chamber.

In one or more embodiments, the engagement mechanism positions the surface of the at least one vessel in a manner that protrudes into the second chamber.

In one or more embodiments, the engagement mechanism positions the surface of the at least one vessel in a manner that is below an inner surface of the second chamber.

In one or more embodiments, the first connection port is covered by a cover. In one or more embodiments, the second connection port is covered by a cover.

In one or more embodiments, the first and/or second chamber(s) is manufactured from a material selected from the group consisting of plastic, rigid plastic, flexible plastic, and combinations thereof.

In one or more embodiments, the device is disposable, intended for one-time use, or repeat use.

In one or more embodiments, the first and/or second chambers comprise a top wall, a bottom wall, and side walls. In one or more embodiments, the first and second chambers share a side wall.

In one or more embodiments, the first and second chambers are disposed next to each other, or one above the other.

In one or more embodiments, the at least one vessel is attached to or integrally attached to the second chamber.

In one or more embodiments, the at least one vessel is permanently/fixedly attached to the second chamber.

In one or more embodiments, at least one vessel forms a unitary structure with the second chamber. In one or more embodiments, the device further comprising a first piercing member configured to pierce through a surface of the at least one vessel.

In one or more embodiments, the device further comprising a second piercing member configured to pierce through a surface of a second vessel.

In one or more embodiments, the device further comprising one or more horizontal channels between the first chamber and the second chamber, the one or more horizontal channels allowing a laminar flow of the sterilizing/disinfecting substance.

In one or more embodiments, the device further comprising a fluid transfer mechanism allowing movement of the sterilizing/disinfecting substance between the first and the second chambers.

In one or more embodiments, the device further comprising a safety mechanism configured to prevent premature entry of the sterilizing/disinfecting substance into the second chamber.

In one or more embodiments, the device further comprising a third chamber configured to receive the sterilizing/disinfecting substance from the second chamber. In one or more embodiments, the device further comprising one or more horizontal channels between the second chamber and the third chamber, the one or more horizontal channels allowing a laminar flow of the sterilizing/disinfecting substance. In one or more embodiments, the device further comprising a fluid transfer mechanism allowing movement of the sterilizing/disinfecting substance between the second and the third chambers.

In one or more embodiments, the device further comprising a safety mechanism configured to prevent premature entry of the sterilizing/disinfecting substance into the third chamber.

In one or more embodiments, the at least one vessel is a bottle, a syringe, an infusion line, a connector, a filter, a manifold, a bag port, a bottle port, a vial port, and combinations thereof.

In one or more embodiments, the device is integrally attached to a vessel.

In one or more embodiments, the device forms a unitary structure with a vessel. In one or more embodiments, the vessel is a bag or a bottle. In one or more embodiments, the vessel is a vial.

In one or more embodiments, the housing abuts a wall of vessel.

In one or more embodiments, at least one of the first and second chambers is flush mounted to a vessel.

In one or more embodiments, at least one of the first and second chambers is surface mounted to a vessel. In one or more embodiments, the vessel is a bag or a bottle. In one or more embodiments, the vessel is a vial.

In a further aspect, the present invention provides a device for decontaminating a surface of at least one vessel, the device comprising: a housing comprising a first chamber configured to accommodate a sterilizing/disinfecting substance; a second chamber configured to connect the at least one vessel; and an entry aperture disposed between the first chamber and the second chamber, wherein the entry aperture configured to provide for a communication between the first and second chambers, such that said second chamber can receive the sterilizing substance from the first chamber and to thereby decontaminate the surface of the at least one vessel.

In yet a further aspect, the present invention provides device for decontaminating a surface of at least one vessel, the device comprising: a housing comprising a first chamber configured to accommodate a sterilizing/disinfecting substance; a second chamber comprising an engagement mechanism configured to allow an engagement with the at least one vessel; and an entry aperture disposed between the first chamber and the second chamber, wherein the entry aperture configured to provide for a communication between the first and second chambers, such that said second chamber can receive the sterilizing substance from the first chamber and to thereby decontaminate the surface of the at least one vessel.

In a further aspect, the present invention provides a method of decontaminating a surface of at least one vessel, the method comprising: providing a decontamination device comprising a first chamber configured to accommodate a sterilizing/disinfecting substance and a second chamber, fluidly connected to the first chamber and configured to receive the sterilizing/disinfecting substance from the first chamber; providing at least one vessel having a surface; connecting the at least one vessel to the second chamber; and transferring the sterilizing/disinfecting substance from the first chamber to the second chamber, thereby decontaminating the surface of the at least one vessel.

In one or more embodiments, the connection of the second chamber to the at least one vessel results in a hermetic seal being formed between the second chamber and the at least one vessel.

In one or more embodiments, the hermetically sealed connection between the second chamber and the at least one vessel is isolated from environmental/contaminated air.

In one or more embodiments, transferring the sterilizing/disinfecting substance to the second chamber results in flushing the second chamber and the surface of the at least one vessel.

In one or more embodiments, the sterilizing/disinfecting substance is transferred to the second chamber via an entry aperture disposed between the first chamber and the second chamber.

In one or more embodiments, the method further comprising actuating an entry actuator to manipulate opening and/or closing of the entry aperture.

In one or more embodiments, the method further comprising removing the sterilizing/disinfecting substance from the second chamber

In one or more embodiments, removing the sterilizing/disinfecting substance from the second chamber occurs via an exit aperture provided on the second chamber.

In one or more embodiments, the method further comprising actuating an exit actuator to manipulate opening and/or closing of the exit aperture.

In one or more embodiments, the second chamber comprises a first connection port configured for coupling to a first vessel, and a second connection port configured for coupling to a second vessel wherein the ports provide a connection between the second chamber and the vessels.

In one or more embodiments, the method further comprising removing the sterilizing/disinfecting substance from the second chamber via the first and/or second connection port.

In one or more embodiments, the at least one of the first and second connection ports are configured to allow evaporation of the sterilizing substance from the second chamber.

In one or more embodiments, the method further comprising allowing a fluid passageway between the first vessel and the second vessel via the first connection port and the second connection port.

In one or more embodiments, the fluid passageway between the first and second vessels is via the second chamber.

In one or more embodiments, the decontamination device is pre-attached to the at least one vessel.

In one or more embodiments, the decontamination device is integrally attached to the at least one vessel.

In one or more embodiments, transferring the sterilizing/disinfecting substance from the first chamber to the second chamber occurs after connecting the at least one vessel to the decontamination device.

In one or more embodiments, the fluid passageway between the first vessel and the second vessel occurs after removing the sterilizing/disinfecting substance from the second chamber.

In one or more embodiments, flushing the second chamber forms a chamber that is sterile.

In one or more embodiments, flashing the second chamber forms a conduit where a fluid communication may be formed between the at least one vessel and the decontamination device.

In one or more embodiments, the at least one vessel is connected to the decontamination device via an engagement mechanism.

In one or more embodiments, the engagement mechanism is selected from a group consisting of a thread mechanism, a luer mechanism, a ratchet teeth/retention member mechanism, and an adhesive mechanism.

In one or more embodiments, connecting the decontamination device to the at least one vessel includes twisting of a thread onto a complementary thread, attaching ratchet teeth to a complementary retention member.

In one or more embodiments, the at least one vessel is selected from a bottle, a syringe, an infusion line, a connector, a filter, a manifold, a bag port, a bottle port, a vial port, and combinations thereof.

In one or more embodiments, the method further comprises transferring the sterilizing substance from the second chamber to the third chamber.

In one or more embodiments, the method involves pushing the sterilizing substance into and out of the first and second chambers.

In a further aspect, the present invention provides a device for decontaminating a surface of at least one vessel, the device comprising: a housing having a first compartment and a second compartment; an entry aperture disposed between the first compartment and the second compartment, the entry aperture configured to provide for a fluidic communication between the first and second compartments; and a substance disposed within the first compartment.

In a further aspect, the present invention provides a device for decontaminating a surface of at least one vessel, the device comprising: a housing having a first compartment and a second compartment; an actuator configured to open an entry aperture; an entry aperture disposed between the first compartment and the second compartment, the entry aperture configured to provide for a fluidic communication between the first and second compartments; and a substance disposed within the first compartment.

In a further aspect, the present invention provides a device for decontaminating a surface of at least one vessel, the device comprising: a housing having a first compartment and a second compartment; an actuator configured to move a substance from the first compartment to the second compartment; an entry aperture disposed between the first compartment and the second compartment, the entry aperture configured to provide for a fluidic communication between the first and second compartments; and a substance disposed within the first compartment.

In a further aspect, the present invention provides a device for decontaminating a surface of at least one vessel, the device comprising: a housing having a first compartment and a second compartment; an engagement mechanism attached to the second compartment, the engagement mechanism configured to engage a vessel to the second compartment; an entry aperture disposed between the first compartment and the second compartment, the entry aperture configured to provide for a fluidic communication between the first and second compartments; and a substance disposed within the first compartment.

In a further aspect, the present invention provides a device for decontaminating a surface of at least one vessel, the device comprising: a first housing having a second housing disposed within the first housing; and at least one engagement mechanism configured to engage at least one vessel.

In one or more embodiments, the device further comprises an exit aperture.

In one or more embodiments, the device further comprises a piercing member.

Unless otherwise defined, all technical or/and scientific terms used herein have the same meaning as commonly understood by one of ordinary skill in the art to which the invention pertains. Although methods and materials similar or equivalent to those described herein can be used in the practice or testing of embodiments of the invention, exemplary methods or/and materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-1E are schematic cross section illustrations demonstrating a dual chamber device for decontaminating medical vessels, according to some embodiments of the invention;

FIGS. 2A-2E are schematic illustrations of the device of the preceding figure which further includes piercing members for puncturing a cover of first and second connection ports and entry and exit apertures, according to some embodiments of the invention;

FIGS. 3A-3B are schematic cross section illustrations of the device of the preceding figures which optionally further includes a piercing member, situated within second chamber, for puncturing a cover of entry aperture, according to some embodiments of the invention;

FIGS. 4A-4C are schematic cross section illustrations of the device of the preceding figures which optionally includes an entry valve and a valve manipulator for controllably opening entry aperture, according to some embodiments of the invention;

FIGS. 5A-5B are schematic cross section illustrations of the device of FIGS. 4A-4C wherein the valve manipulator is situated in second chamber, according to some embodiments of the invention;

FIG. 6 is a schematic cross section illustration of the device of the preceding figures which optionally includes an entry valve and an exit valve, according to some embodiments of the invention;

FIG. 7 is a schematic cross section illustration of the device of the preceding figures which optionally includes a filter in first chamber for releasing air pressure, according to some embodiments of the invention;

FIG. 8 is a schematic cross section illustration of the device of the preceding figures which optionally includes a filter in second chamber for releasing air pressure, according to some embodiments of the invention;

FIGS. 9A-9D are schematic cross section illustrations of the device of the preceding figures which optionally or alternatively includes a sponge in second chamber for absorbing the sterilizing substance, thereby allowing fluid passageway between vessels which is devoid of any residuals of said substance, according to some embodiments of the invention;

FIG. 10 is a schematic cross section illustration of the device of the preceding figures which optionally includes an engagement mechanism in the form of a thread, allowing secure and safe engagement between a vessel and second chamber, according to some embodiments of the invention;

FIG. 11 is a schematic cross section illustration of the device of the preceding figures which optionally includes an engagement mechanism in the form of a clamping arms, allowing secure and safe engagement between a vessel and second chamber, according to some embodiments of the invention;

FIG. 12 is a schematic cross section illustration of the device of the preceding figures which optionally includes an engagement mechanism in the form of a sliding rail, allowing secure and safe engagement between a vessel and second chamber, according to some embodiments of the invention;

FIG. 13 is a schematic cross section illustration of the device of the preceding figures which optionally includes an engagement mechanism in the form of a retention member, allowing secure and safe engagement between a vessel and second chamber, according to some embodiments of the invention;

FIGS. 14A-14B are schematic cross section illustrations of the device of the preceding figures which optionally or alternatively includes a sterilizing substance transfer mechanism in the form of a moveable shaft, according to some embodiments of the invention;

FIGS. 15A-15C are schematic cross section illustrations of the device of FIGS. 14A-14B which additionally includes a handle operable to manipulate movement of the moveable shaft, according to some embodiments of the invention;

FIGS. 16A-16B are schematic cross section illustrations of the device of FIGS. 15A-15C which additionally includes a plurality of entry aperture valves and a plurality of exit aperture valves, according to some embodiments of the invention;

FIGS. 17A-17B are schematic cross section illustrations of the device of FIGS. 14A-14B which additionally includes a spring operable to manipulate movement of the moveable shaft across first chamber, according to some embodiments of the invention;

FIGS. 18A-18B are schematic cross section illustrations of the device of FIGS. 14A-14B which additionally includes a safety mechanism for preventing premature transfer of the sterilizing substance, according to some embodiments of the invention;

FIGS. 19A-19B are schematic cross section illustrations of the device of the preceding figures which further includes an elongated entry channel between first chamber a second chamber, according to some embodiments of the invention;

FIGS. 20A-20B are schematic cross section illustrations of the device of the preceding figures which further includes a plurality of elongated exit channel extending from second chamber, according to some embodiments of the invention;

FIGS. 21A-21B are schematic cross section illustrations of the device of the preceding figures which further includes a plurality of elongated entry channels extending from first chamber into second chamber, according to some embodiments of the invention;

FIGS. 22A-22E are schematic cross section illustrations of the device of the preceding figures which further includes a third chamber for accommodating the sterilizing substance following decontamination of the second chamber and vessels attached thereto, according to some embodiments of the invention;

FIGS. 23A-23E are schematic cross section illustrations of the device of FIGS. 22A-22E which further includes a first sterilizing substance transfer mechanism in the form of a moveable shaft which pushes the sterilizing substance into the second chamber and a second sterilizing substance transfer mechanism in the form of a moveable shaft which pulls the sterilizing substance into the third chamber, according to some embodiments of the invention;

FIG. 24 is a schematic cross section illustration of the device of FIGS. 22A-22E which further includes an entry valve and an exit valve, according to some embodiments of the invention;

FIGS. 25A-25E are schematic cross section illustrations of the device of FIGS. 22A-22E which further comprises a safety mechanism for preventing premature transfer of the sterilizing substance, according to some embodiments of the invention;

FIGS. 26A-26D are schematic cross section illustrations of the device of FIGS. 22A-22E which further comprises a locking mechanism configured to restrict movement of the sterilizing substance transfer mechanism, according to some embodiments of the invention;

FIGS. 27A-27E are schematic cross section illustrations of yet a further exemplary device which includes a first and a second chambers being collapsible, thereby allowing transferring the sterilizing substance from the first chamber to the second chamber and from the second chamber out of the device, according to some embodiments of the invention;

FIGS. 28A-28D are schematic cross section illustrations of yet a further exemplary device which includes a first and a second chambers being collapsible, wherein the second chamber is disposed within the first chamber, according to some embodiments of the invention;

FIG. 29 is a schematic cross section illustration of yet a further exemplary device which includes a plurality of connection ports for connecting a plurality of vessels, according to some embodiments of the invention.

It should be appreciated that for simplicity and clarity of illustration, elements shown in the figures have not necessarily been drawn to scale. For example, the dimensions of some of the elements are exaggerated relative to each other for clarity. Further, where considered appropriate, reference numerals have been repeated among the figures to indicate corresponding elements.

### DETAILED DESCRIPTION OF THE INVENTION

It is understood that the invention is not limited to the particular methodology, devices, items or products etc., described herein, as these may vary as the skilled artisan will recognize. It is also to be understood that the terminology used herein is used for the purpose of describing particular embodiments only and is not intended to limit the scope of the invention. The following exemplary embodiments may be described in the context of exemplary decontamination devices and systems for ease of description and understanding. However, the invention is not limited to the specifically described devices, systems and methods and may be adapted to various applications without departing from the overall scope of the invention. All ranges disclosed herein include the endpoints. The use of the term “or” shall be construed to mean “and/or” unless the specific context indicates otherwise.

The present invention pertains to devices, methods and systems allowing the decontamination of, and fluid passageway between, vessels. The herein disclosed devices, methods and systems are particularly useful for medical purposes, wherein sterile preparations of medical substances are vastly needed. In one embodiment, the present invention provides systems configured to decontaminate one or more surfaces of a vessel(s). In one embodiment, the present invention provides systems configured to allow a substantially decontaminated engagement between vessels. In one or more embodiments, the systems of the invention allow a substantially decontaminated fluid transfer between the one or more vessel(s).

The invention provides a solution to an unmet and long felt need in the medical settings and allows connecting in a sterile manner, two or more vessels. The herein disclosed devices and systems are user friendly, cost effective and abolish the need for complicated and expensive equipment and known methods for transferring medical substances.

The devices and systems of the invention are based on a dual chamber/compartment or a triple chamber/compartment device that includes a sterilizing substance that can be transferred between the chambers and thereby flush the inner surfaces of the chambers. By flushing the inner walls of a chamber, the surface(s) of a vessel connected to and exposed to a flushed chamber can also be flushed and decontaminated. The sterilizing substance can thereafter exit the flushed chamber through dedicated aperture(s) and allow for a subsequent establishment of a fluid passageway between vessels connected to the decontaminated chamber/compartment. In one or embodiments, the device of the invention includes a third chamber for accommodating the sterilizing substance after flushing the second chamber. The third chamber allows a repeated use of the device and the sterilizing substance such that the sterilizing substance may be moved from the third chamber back to the second chamber to allow subsequent flushing of the second chamber and new vessels attached thereto.

Thus, in an aspect of the invention, there is provided a device for decontaminating a surface of at least one vessel, the device comprising: a housing comprising a first chamber configured to accommodate a sterilizing/disinfecting substance; and a second chamber, connected to the first chamber and configured to connect the at least one vessel, the second chamber further configured to receive the sterilizing substance from the first chamber and to thereby decontaminate the surface of the at least one vessel.

The herein disclosed device includes two or more chambers, wherein a first chamber includes a sterilizing substance and a second chamber is configured to receive the sterilizing substance from the first chamber. The second chamber is further configured to engage with one or more vessels. The surface(s) of the one or more vessel(s) connected to the second chamber can be decontaminated by the sterilizing substance following transferring same from the first chamber to the second chamber. The sterilizing substance may be subsequently removed from the second chamber via a dedicated exit aperture and fluid passageway between the vessels connected to the second chamber may be established.

In an embodiment of the invention, the herein disclosed devices and systems are disposable, intended for one-time use. In an embodiment of the invention, the herein disclosed devices and systems are non-disposable, intended for repeated use.

In one or more embodiments, the device is conveniently light presenting a weight of no more than about 50 grams (gr). For example, less than about 40 gr, less than about 30 mm gr, less than about 20 gr, less than about 10 gr, or less than about 5 gr.

The device may be transparent allowing visualization of its interior, or opaque.

The device may be a closed chamber which forms a tight, optionally, airtight connection between the vessels. The device may be a closed chamber which forms an interior which is hermetically sealed/isolated from surrounding/environmental air.

The dual or triple chamber device of the invention may be manufactured from various materials. The one or more chambers may be made from a flexible or from a rigid material. Suitable materials include, without limitation a plastic, a glass, a rigid plastic, a flexible plastic, an elastomeric material and combinations thereof. In one or more embodiments, the herein disclosed device has a height, width, and/or depth) of up to about 5 inches. For example, up to about 4 inches, up to about 3 inches, up to about 2 inches, up to about 1 inches, up to about ½ inch in height, width, and/or depth.

In one or more embodiments, the herein disclosed device has a weight of up to about 150 grams. For example, up to about 100 grams, up to about 50 grams, up to about 25 grams, up to about 10 grams, or up to about 5 grams.

In one or more embodiments, the one or more chambers of the herein disclosed device has a volume of less than 100 milliliters (ml). For example, less than 50 ml, less than 25 ml, less than 10 ml, less than 5 ml, less than 3 ml, 2 ml, 1 ml, 0.5 ml, 0.25 ml, or less than 0.1 ml.

In one or more embodiments, the term “sterilizing substance” encompasses any substance that can eliminate, remove, deactivate and/or reduce the presence of contaminates, such as, microorganisms, air particles, or any of the alike. In one embodiment, the term “sterilizing substance” is interchangeable with the term “disinfecting substance”. In one or more embodiments, the sterilizing substance may be chosen from, without limitation, an antibacterial substance, an antiviral substance, an anti-tuberculin substance, and a combination thereof. Exemplary sterilizing substances include without limitation an alcohol (e.g., ethanol and isopropanol), an aldehyde (e.g., glutaraldehyde, and formaldehyde), an oxidizing agent (e.g., hydrogen peroxide), an acid (e.g., peroxy acid), a phenol, chlorine, guanidinium thiocyanate, sodium hydroxide, a quaternary ammonium compound, and a combination thereof. In one or more embodiments, the sterilizing substance may be, but is not limited to, an antibacterial substance, an antiviral substance (e.g., an anti-HIV agent), an anti-tuberculin substance, an anti-fungal, and combinations thereof. In one or more embodiments, the sterilizing substance may be, but is not limited to, a bactericidal substance, a virucidal substance, a fungicidal substance, and combinations thereof. In one or more embodiments, the sterilizing substance may be an inert substance. In one or more embodiments, the sterilizing substance may be water. In one or more embodiments, the water may be sterile water. The sterilizing substance may be a liquid, a gas, or a semi-solid substance. The sterilizing substance may optionally be provided within the device when in a pressurized state. The pressure differences between the chambers optionally allows movement of the sterilizing substance from a highly pressurized location (e.g., the first chamber, or the first and/or second chamber when three chambers are provided) to a lower pressurized location (e.g., the second chamber or the second and/or third chamber when three chambers are provided).

In one or more embodiments, the one or more of the surfaces(s) of the vessel(s) is flat and/or smooth. The surface may be substantially flat or partially flat or sufficiently flat, allowing efficient decontamination thereof by the sterilizing substance.

The dual or triple chamber device of the invention may optionally include one or more aperture(s), optionally covered by a filter, a cover or are sealed by a valve. The apertures may be used for allowing passage of the sterilizing substance between the chambers and/or removal of the sterilizing substance. The dual or triple chamber device of the invention may optionally include one or more port(s), optionally covered by a filter, a cover or are sealed by a valve. The ports provided to allow for an engagement with medical vessels may be sealed or covered by a frangible seal. In one or more embodiments, when a valve is provided between a first and a second chamber, the valve allows movement of the sterilizing substance from the first chamber to the second chamber. In one or more embodiments, the valve prevents backflow of the sterilizing substance from the second chamber back into the first chamber. In one or more embodiments, when a valve is provided within the exit aperture, the valve is thus configured to allow removal of the sterilizing substance via the exit aperture. In one or more embodiments, the valve prevents backflow of the sterilizing substance back into the second and/or third chamber.

In one or more embodiments, a filter provided within, on, or in the housing, such as within the first, second, and/or third chamber, may be a sterilizing filter. In one or more embodiments, the filter may be disposed in an aperture of a wall of the housing of the device. For example, a filter may be disposed covering an aperture of a wall of the device. In one or more embodiments, the filter may be disposed within or covering an aperture of the first, second, and/or third chamber. In one or more embodiments, the filter may have a porosity of 5 microns or less. For example, the filter may have pores with a diameter of less than 4 microns, less than 3 microns, less than 2 microns, or less than 1 micron. In one or more embodiments, the filter has a porosity of 0.22 microns or less. In one or more embodiments, the filter covers an aperture of the first chamber of the housing. In one or more embodiments, the filter covers an aperture of the second chamber of the housing. In one or more embodiments, the filter covers an aperture of the third chamber of the housing. In one or more embodiments, the filter is disposed in or covers an aperture of the first chamber of the housing and a second filter is disposed in or covers an aperture of the second chamber of the housing. In one or more embodiments, the filter allows for the evaporation of the sterilizing substance from the first and/or second and/or third chamber.

In one or more embodiments, the device of the invention is provided when the device is already connected to one or more medical vessels. In accordance with this embodiment, the one or more medical vessels may be integrally manufactured with the device and provided or distributed to consumers as such. In one or more embodiments, the device forms a unitary structure with at least one medical vessel. In one or more embodiments, the medical vessel is a container. In one or more embodiments, the container is a bag, a bottle, or a syringe. In one or more embodiments, the medical vessel is a connector configured to connect the device to a second medical vessel. In one or more embodiments, the vessel is a filter or a buretrol.

In accordance with this embodiment, the device may be configured to allow a connection to additional one or more vessel(s). Alternatively, the device of the invention is provided without any attachment to a vessel and is configured to connect a plurality of vessels and decontaminate thereof. In one or more embodiments, the at least one vessel ranges from one vessel to ten vessels, wherein a surface of at least one or each of the vessel(s) is configured to be decontaminated by the sterilizing substance.

In one or more embodiments, the device includes a plurality of chambers or compartments. Optionally, the chambers are disposed one parallel or next or adjacent each other, providing a left chamber and a right chamber. Optionally, the chambers are disposed one above the other, providing a top chamber and a bottom chamber.

In one or more embodiments, the second chamber has an engagement mechanism configured or adapted to connect the at least one vessel to the chamber. Various types of engagement mechanisms may be applicable and are contemplated. For example, the engagement mechanism is selected from, but is not limited to the group consisting of: a thread, a luer, a ratchet teeth mechanism, a sliding rail mechanism, a retention mechanism, a clamping mechanism and an adhesive mechanism. In an embodiment of the invention, the engagement mechanism positions the surface of the at least one vessel at about same level as an interior wall of the chamber. In an embodiment of the invention, the engagement mechanism positions the surface of the vessel at the same level as an interior wall of a chamber of the system. In an embodiment of the invention, the engagement mechanism positions the surface of the at least one vessel in a manner that protrudes into the chamber. In an embodiment of the invention, the protruding of the surface of the at least one vessel is a slight/minor protruding. In an embodiment of the invention, the protruding of the surface of the at least one vessel ranges from less than one eighth of an inch to less than half an inch. In an embodiment of the invention, the engagement mechanism positions the surface of the at least one vessel in a manner that is below an inner surface of the chamber. In an embodiment of the invention, the positioning of the surface of the at least one vessel ranges from less than one eighth of an inch to less than a half an inch from the inner surface of the chamber. In an embodiment of the invention, the engagement mechanism positions the surface of the at least one vessel slightly below an inner wall/surface of the chamber while allowing the sterilizing substance to reach the entire surface and decontaminate the entire surface of the at least one vessel.

In one or more embodiments, the herein disclosed invention allows transferring medical substances in a contaminant-free, or in a substantially contaminant-free manner.

In one or more embodiments, the herein disclosed invention affords an engagement of vessels in a contaminant-free, or in a substantially contaminant-free manner.

In one or more embodiments, the herein disclosed invention provides a fluidic passageway or communication between vessels in a contaminant-free, or in a substantially contaminant-free manner. The fluid passageway between vessels may be via the second chamber.

In one or more embodiments, the herein disclosed invention, allows to substantially decrease the chances to introduce contaminants within a medical substance, when preparing medical substances for administration to patients.

As used herein the term “medical substances” refers to various types of materials that should be kept sterile. The medical substances may be liquid, semi-solid, or gas. The term medical substances may refer to and encompasses any of the various pharmaceutical drugs, fluids, nutritional products and the like. In an embodiment of the invention the term “medical substance” is interchangeable with the term “pharmaceutical substance.”

As used herein the term “contaminant-free” is interchangeable with the term “sterile”, “disinfected”, and “decontaminated”. The term refers to substances that are free or substantially free of ambient air particles and/or pathogens and/or micro-organisms, and/or any of the alike. Typically, when less or no air is introduced within medical substances, the chances of contamination by pathogens, such as, bacteria, viruses, funguses, spores, pyrogens or the alike is completely abolished or significantly reduced.

As used herein, the term “substantially contaminant-free” means significantly less ambient air present when transferring medical substances with the herein disclosed vessels and systems, as compared to comparable conditions for transferring medical substances without the herein disclosed vessels and systems.

As used herein the term “ambient air particles” is interchangeable with the term “environmental air particles” and refers to air particles present in a non-filtered environment. The air particles may contain dirt, such as dust. Alternatively, or additionally, the air particles may contain pathogens or other microorganisms, or any of the alike.

As used herein the term “vessel” refers to any device utilized for containing substances as herein disclosed. In one or more embodiments, the vessels may be used for containing medical substances. In an embodiment of the invention, the vessel is a medical vessel. In one or more embodiments, the term “medical vessel” is interchangeable with the term “pharmaceutical vessel”. In an embodiment of the invention, the vessel is a medical device. In an embodiment of the invention, the vessels are used for, and adapted to allow connection to another vessel or decontamination device. In an embodiment of the invention, the vessel is utilized for containing a medical substance. Various types of vessels are contemplated. The vessel may be selected, without limitation, from a vial, a bag, a chamber, a bottle, and the alike. In an embodiment of the invention, the term vessel further encompasses elements that can be used to connect between vessels. In accordance with this embodiment, the vessel may be selected, without limitation, from a connector, a syringe, an infusion line, a tubing, a syringe, a filter, a port, a buretrol and a manifold.

As used herein the term “fluid communication” refers to two or more vessels in which substances may pass therethrough either directly or indirectly. The fluid communication may occur via a fluid passageway that allows for the flow/transfer of substances. In an embodiment of the invention, fluid passageway between vessels is established when any seals or covers of the ports of the herein disclosed systems and/or of the vessels are open. Optionally, the vessels and/or ports and/or apertures of the herein disclosed devices are provided with seals/covers and those seals/cover may optionally become open upon piercing thereof, for example, by a piercing member. Optionally, the vessels and/or ports and/or apertures of the herein disclosed devices are provided with seals/covers and those seals/cover may optionally, alternatively or additionally be opened upon uncovering or removing thereof by a user. Optionally, one or more of the seals/covers disclosed herein are fragile, frangible, breakable, pierceable, and/or displaceable allowing piercing thereof with a sharp instrument (i.e., a piercing member). The piercing member may optionally be a needle, for example, a hollowed needle, but other sharp elements are contemplated.

In one or more embodiments, a one or more piercing members may be configured to pierce through a surface of a vessel to thereby establish a fluidic communication between vessels. Optionally, the one or more piercing members is actuated to pierce through a surface of a vessel by a button/actuator, which may be operated by a twisting and/or turning and/or a pushing motion. Optionally, a twisting and/or turning and/or a pushing motion of the device relative to the vessel allows to pierce through a surface of a vessel.

In an embodiment of the invention, the herein disclosed devices, systems and methods allow fluid communication in a contaminant-free, or in a substantially contaminant-free manner. For example, between three or more, four or more, five or more or six or more vessels. In an embodiment of the invention, the herein disclosed devices, systems and methods allow fluid communication between three, four, five six, or seven vessels. For example, the devices, systems, and methods of the invention allow fluid communication between two bags, between a vial and a bag, between a syringe and a bag, between a syringe and a vial, between a connector and a bag, between a connector and a vial or between a syringe and a connector. In one or more embodiments, the herein disclosed device(s) may be flush mounted to a vessel. In one or more embodiments, the herein disclosed device(s) may be flush mounted to a wall of a vessel. In one or more embodiments, the herein disclosed device(s) may be surface mounted to a vessel. In one or more embodiments, the herein disclosed device(s) may be surface mounted to a wall of a vessel. In one or more embodiments, the herein disclosed device(s) may abut a vessel.

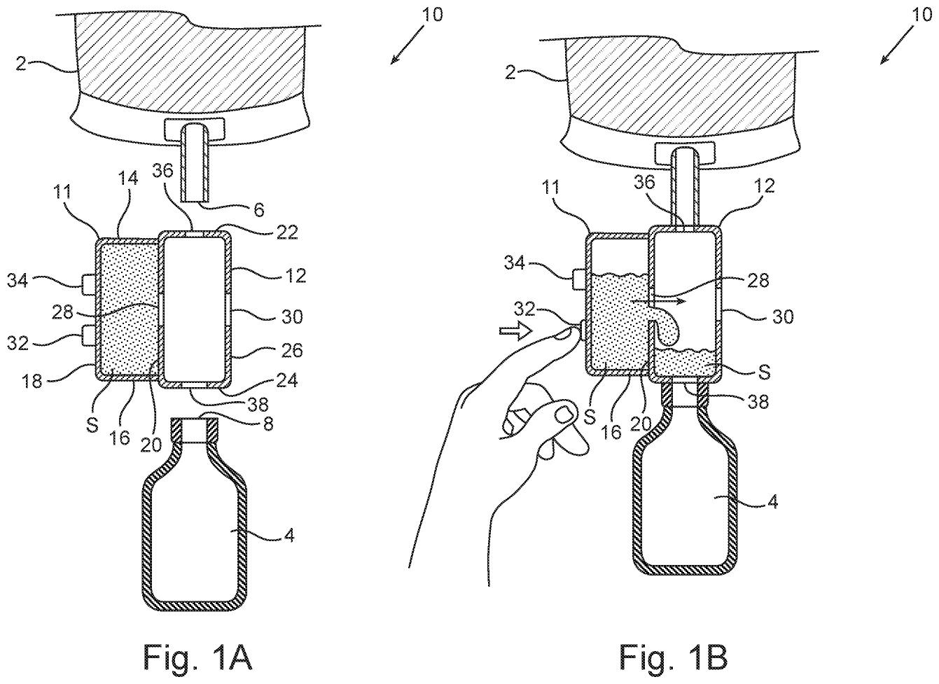

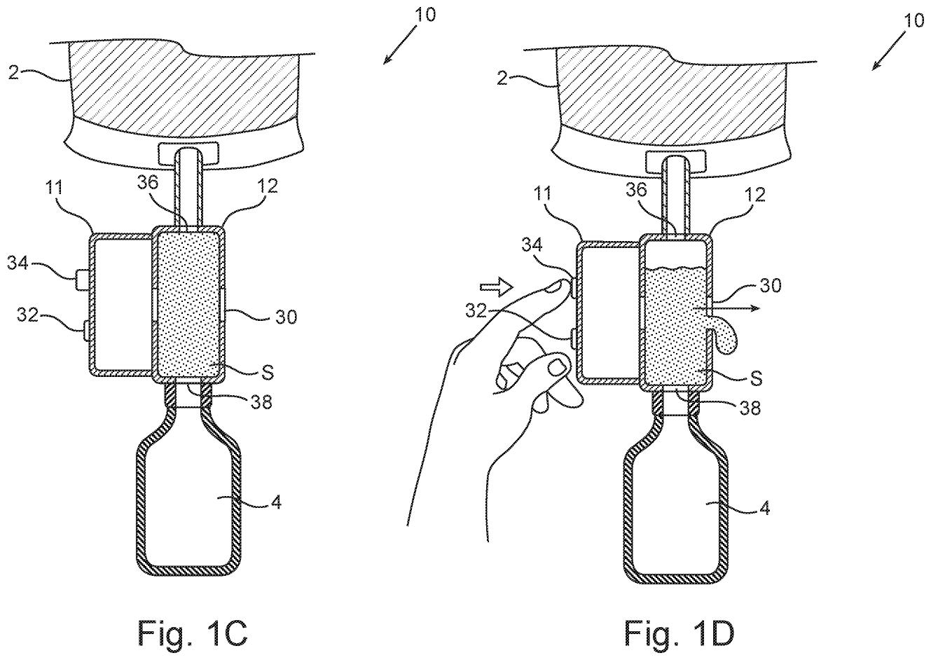

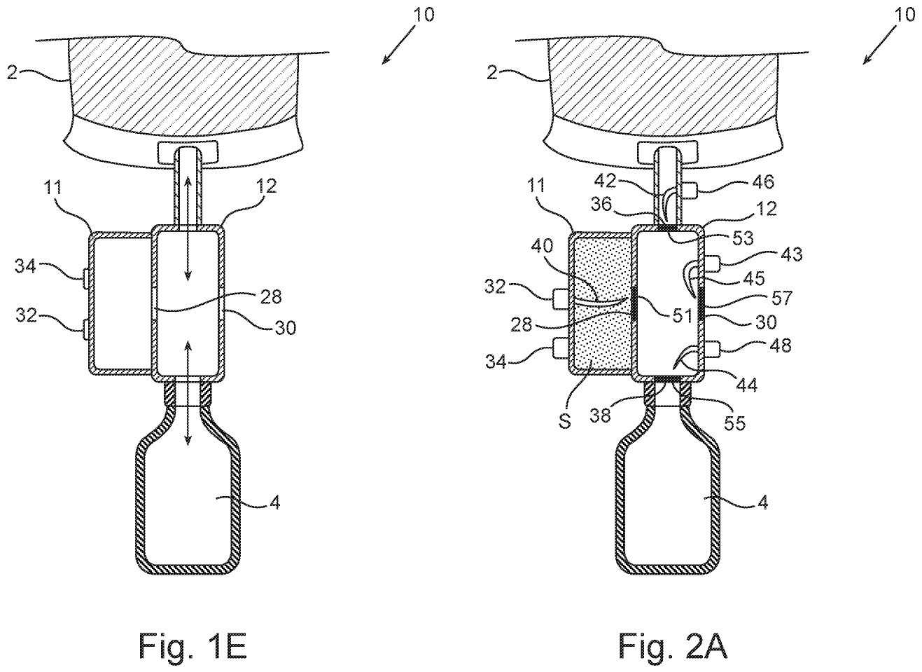

Referring now to the drawings, FIGS. 1A-1E illustrate device 10 for decontaminating a surface 6 of a first vessel 2 and a surface 8 of a second vessel 4. The device 10 includes a first chamber 11 and a second chamber 12. First chamber 11 is configured to accommodate a sterilizing/disinfecting substance S. Second chamber 12, is fluidly connected to the first chamber 11 and configured to connect vessels 2 and 4 and to receive the sterilizing substance S from the first chamber 11. By receiving sterilizing substance S from the first chamber 11, second chamber 12 facilitates decontamination of the surfaces 6 and 8 of vessels 2 and 4, respectively.

First chamber 11 includes a top wall 14, a bottom wall 16, and side walls 18 and 20. Second chamber 12 includes a top wall 22, a bottom wall 24, and side walls 20 and 26. As can be seen in the figure, first and second chambers 11 and 12, respectively, disposed one next to the other and share side wall 20.

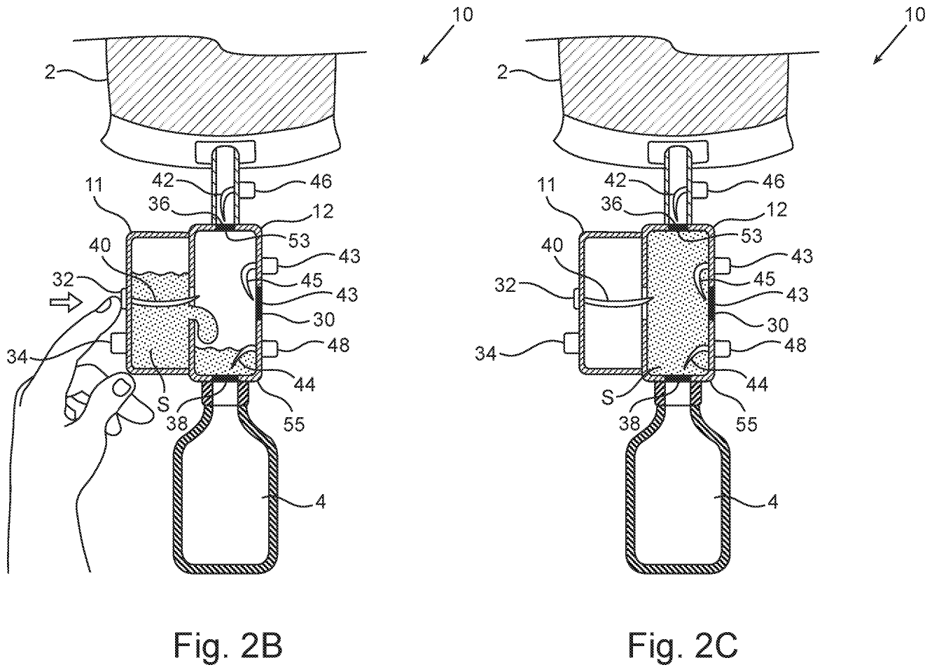

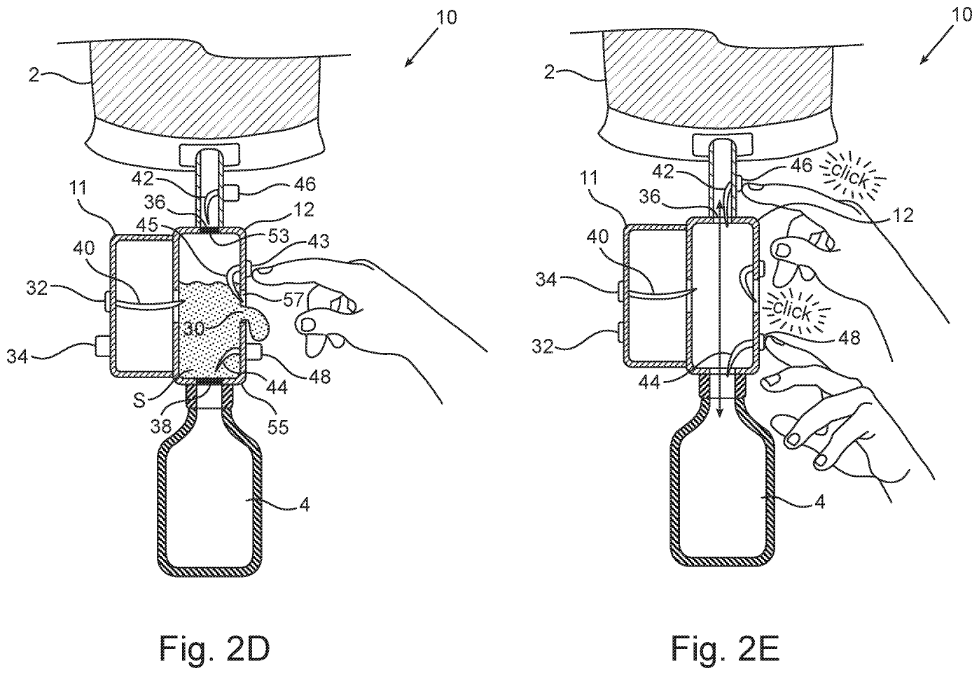

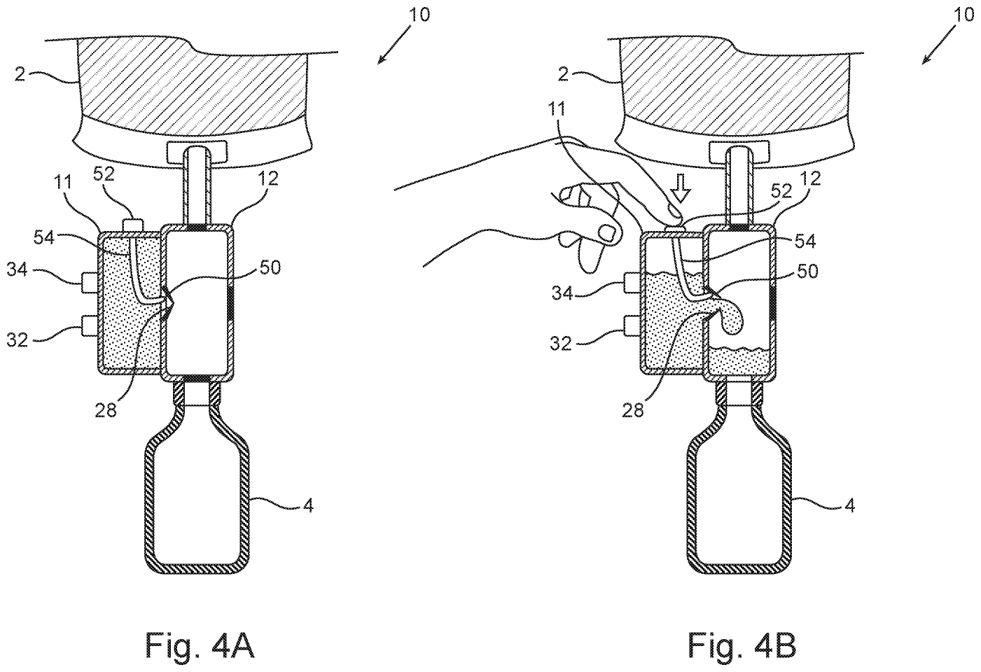

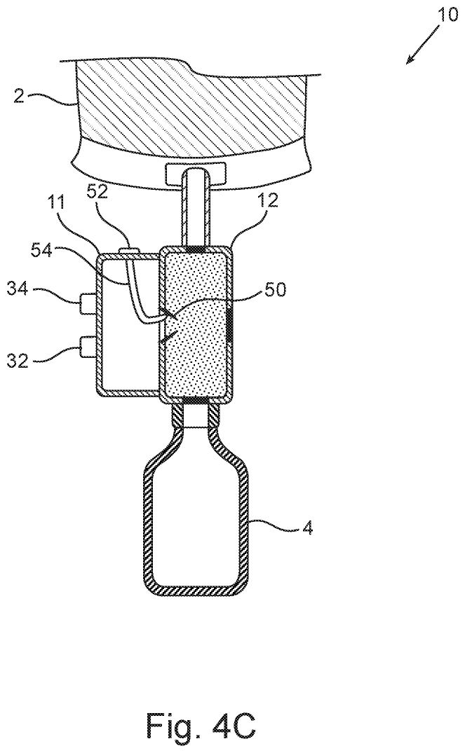

Device 10 further includes an entry aperture 28 disposed between first chamber 11 and second chamber 12, the entry aperture 28 is configured to allow the sterilizing/disinfecting substance S to enter or flow from the first chamber 11 to the second chamber 12. In one or more embodiments, entry aperture 28 is sealed. Optionally, entry aperture 28 is sealed by a cover (see for example FIGS. 2A-2D). Alternatively, or additionally, entry aperture 28 is sealed or covered by an entry valve, optionally a one-way (see for example FIGS. 4A-4C).

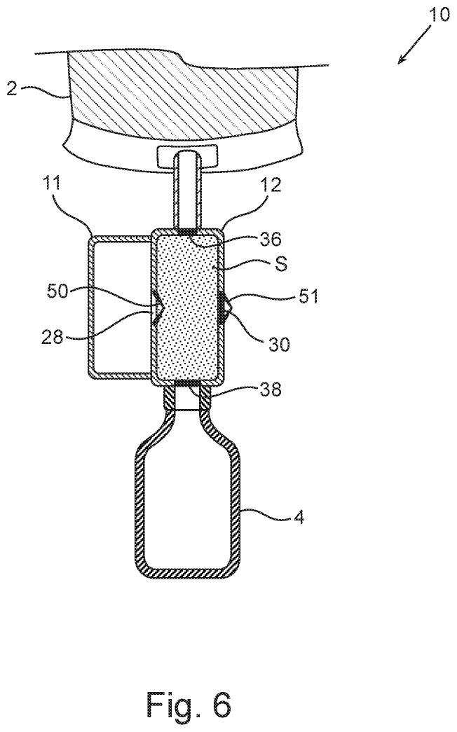

Second chamber 12 includes an exit aperture 30 for allowing removal of the sterilizing/disinfecting substance S from the second chamber 12. Similarly, to entry aperture 28, exit aperture 30, may according to one or more embodiments, be sealed. Optionally, exit aperture is sealed by a cover (see for example FIGS. 2A-2D). Alternatively, or additionally, exit aperture 30 includes an exit valve, optionally a one-way (see for example FIG. 6).

Device 10 further includes one or more actuators operable to manipulate opening and closing of the entry aperture 28 and/or the exit aperture 30. In an exemplary embodiment, device 10 includes an entry actuator 32 configured to manipulate opening and/or closing of the entry aperture 28 and an exit actuator 34 configured to manipulate closing and/or opening of exit aperture 30. The one or more actuators may be connected to the first chamber and/or the second chamber an exterior surface thereof. Optionally, in case there is a seal or a cover along the entry aperture 28 and/or the exit aperture 30, the action of actuating actuator 32 and/or 34, respectively, allows opening of the seal or cover (FIGS. 2A-2E). Optionally, in case there is a valve (see FIGS. 4A-4C, and 6) along the entry aperture 28 and/or the exit aperture 30, the action of actuating actuator 32 and/or 34, respectively, allows opening the valve.

Actuators 32 and 34 may be mounted, but not necessarily, to the first chamber 11, optionally, from an exterior surface thereof. Alternatively, optionally or additionally, actuators 32 and 34 may be mounted to the second chamber 12, optionally, from an exterior surface thereof. Various types of entry actuator 32 and exit actuator 34 are contemplated. For example, the entry actuator 32 and exit actuator 34 may each present a form, selected from, but not limited to, a handle, a tab, and a button. The entry actuator and exit actuator may be located in or on first or second or any other compartment or chamber on or within the device. Entry actuator 32 may be connected to entry aperture 28 to enable the opening of entry aperture 28. The connection between entry actuator 32 and entry aperture 28 may facilitated by a connecting element (not shown) disposed within the device. Exit actuator 34 may be connected to exit aperture 30 to enable the opening of exit aperture 30. The connection between exit actuator 34 and exit aperture 30 may be facilitated by a connecting element (not shown) disposed within the device.

Second chamber 12 further includes a first connection port 36 configured for coupling to vessel 2. First connection port 36 provides a connection between the second chamber 12 and the first vessel 2. Second chamber 12 further includes a second connection port 38 configured for coupling to second vessel 4. Second connection port 38 provides a connection between the second chamber 12 and the second vessel 4.

Optionally, first vessel 2 or second vessel 4 is provided when already attached to or integrally attached to second chamber 12. Vessels 2 and/or 4 may be provided when permanently/fixedly attached to second chamber 12. Device 10 is configured to attach and decontaminate various types of vessels. The vessel may be any of the known vessels used in the medical settings, including, but not limited to a bag, a bottle, a vial, a syringe, an infusion line, a connector, a filter, a manifold, a bag port, a bottle port, a vial port, a buretrol and combinations thereof.

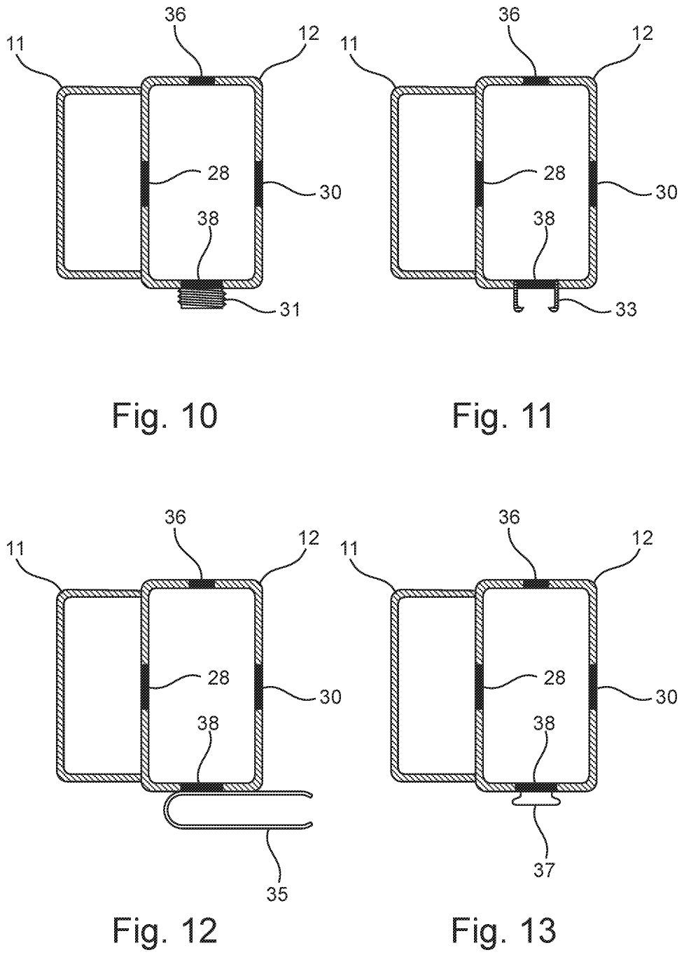

Various attachment or engagement mechanisms between first vessel 2 and second vessel 4 and the herein disclosed device are contemplated. For example, a thread 31 can be connected to device 10 (FIG. 10) and engaged with a complementary thread on first or second vessel. Optionally, a clamping mechanism that includes clamping arms 33 can be connected to device 10 (FIG. 11) and engaged with first or second vessel. Further optionally, clamping rail mechanism 35 may be used (FIG. 12) to attach a vessel to device 10. In this clamping rail mechanism 35, the vessel is firstly connected to the clamping rails 35 and slides along until reaching an end point allowing connection to a connection port on or within device 10 (i.e., first connection port 36 or second connection port 38). Further optionally, a retention member 37 (FIG. 13) may be used to allow an engagement between device 10 and a vessel.

FIG. 1A depicts device 10 when the sterilizing substance S is enclosed within first chamber 11. FIG. 1B depicts entrance of sterilizing substance S, via entry aperture 28, into second chamber 12. By entrance into the second chamber 12, the sterilizing substance S flushes chamber 12 and also ports 36 and 38 and surfaces 6 and 8 via which vessels 2 and 4 are attached to second chamber 12. The flush of the second chamber 12 facilitates decontamination of surfaces 6 and 8 of vessels 2 and 4. The entry of the sterilizing substance S is actuated by a user upon manipulation of entry actuator 32 to open entry aperture 28. The first connection port 36 and second connection port 38 and further second chamber 12 are configured to allow a fluid passageway between the first vessel 2 and the second vessel 4. FIG. 1C depicts second chamber 12 following entry of the sterilizing substance S thereto. By entrance of the sterilizing substance S into the second chamber 12 and fill thereof, surfaces 6 and 8 of the vessels 2 and 4 are flushed, cleaned and decontaminated. The sterilizing substance S can then be removed from second chamber 12 upon manipulation of exit actuator 34 to open exit aperture 30 (FIG. 1D). At FIG. 1E, vessels 2 and 4 are shown wherein a fluid passageway is formed therebetween and a medical substance or any other fluid freely flows from vessel 2 towards vessel 4 or from vessel 4 to towards vessel 2 via second chamber 12. In one or more embodiments, the inner volume of second chamber 12 may be less than about 1 milliliter.

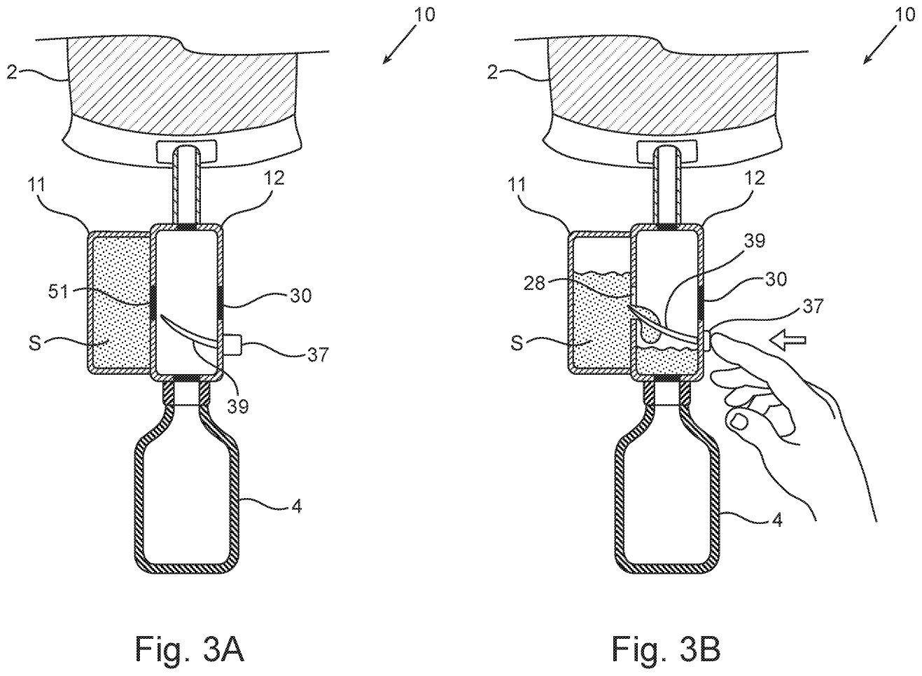

FIGS. 2A-2E illustrate device 10, wherein entry aperture 28, exit aperture 30, first connection port 36 and second connection port 38 are sealed by covers 51, 57, 53, and 55, respectively, which may be displaceable and/or frangible, and/or puncturable. The device 10 further includes a first piercing member 40 configured to rupture or pierce entry aperture cover 51, optionally, upon actuating entry actuator 32. First piercing member 40 is shown located within first chamber 11, but it can be positioned in other locations within device 10 as long as it is configured to pierce entry aperture cover 51. For example, FIGS. 3A-3B, demonstrate a piercing member 39 positioned in second chamber 12 such that piercing of cover 51 is performed from within chamber 12. Upon pressing piercing member actuator 37 by a user, the piercing member 39 is operable to pierce cover 51, thereby allowing sterilizing substance S to flow or move from first chamber 11 to second chamber 12 and decontaminate thereby a surface of vessels 2 and 4.

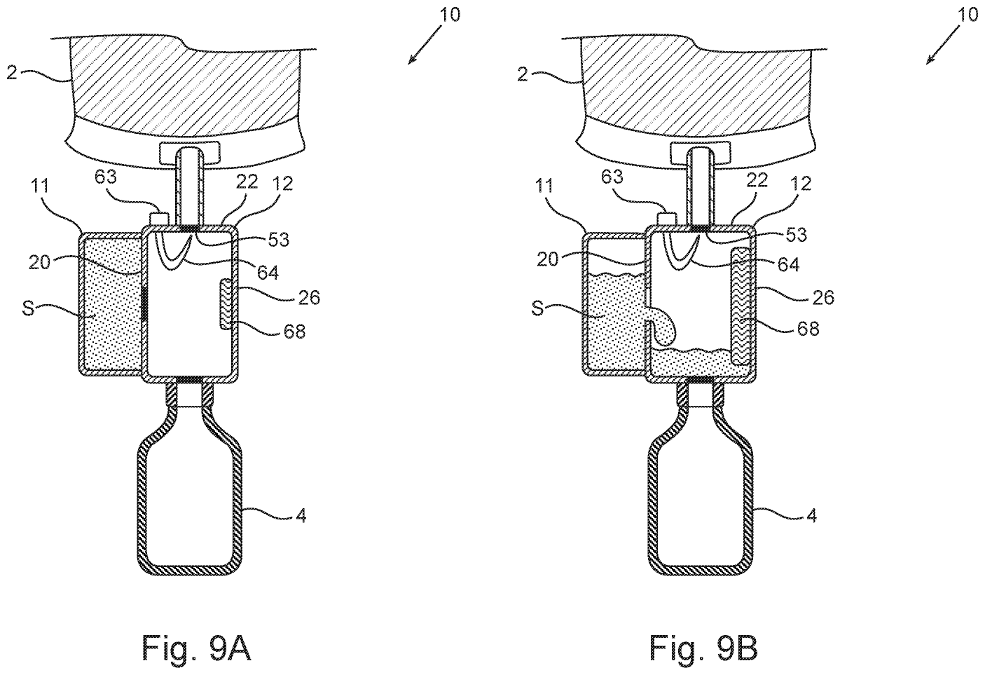

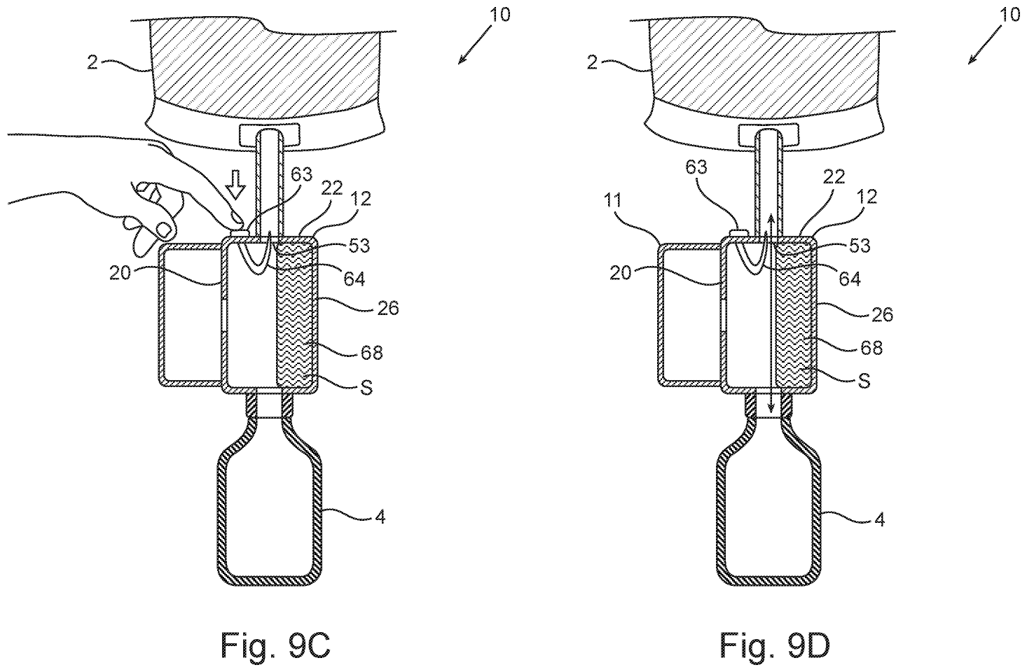

Referring back to FIGS. 2A-2E, second piercing member 45 and actuator 43 are provided to puncture or open cover 57 of exit aperture 30. A third piercing member 42 and a forth piercing member 44 are provided to rupture or pierce covers 53 and 55 of first connection port 36 and second connection port 38, upon actuating actuators 46 and 48, respectively. Third piercing member 42 is shown when located within vessel 2 or a port thereof but it can be positioned in other locations, for example, as shown in FIGS. 9A-9D, piercing member 64 and an actuator 63 thereof can be located in chamber 12. Piercing member 64 extends from top wall 22 and has a curvature such that a sharp edge thereof points towards cover 53 and can puncture said cover 53 upon actuating actuator 63. Similarly, various positions are contemplated for fourth piercing member 44 and actuator 48 thereof, as long as piercing of cover 55 is allowed. Optionally, third piercing member 42 and/or fourth piercing member 44 puncture, or rupture or pierce covers 53 and 55 at about the same time or at different times sterilizing substance S enters into second chamber 12.

FIG. 2A illustrates device 10 wherein the sterilizing substance S enclosed within first chamber 11. In FIG. 2B entry actuator 32 is activated to allow piercing of cover 51 by first piercing member 40. Sterilizing substance S is allowed to enter into second chamber 12. FIG. 2C illustrates sterilizing substance S disposed in second chamber 12 and the surfaces of vessels 2 and 4 that engage device 10 being decontaminated by the sterilizing substance S. In FIG. 2D, the actuator 43 is operated to pierce cover 57 and allow removal of sterilizing substance S from chamber 12. In FIG. 2E, the actuators 46 and 48 are operated, optionally by a pressing motion to allow piercing members 42 and 44 to pierce or rupture covers 53 and 38, respectively. As a result, a fluid passageway can be formed between first vessel 2 and second vessel 4. Optionally, one or more of the above mentioned piercing members 40, may be operable to puncture a cover or seal via a push, and/or a twist (i.e., turning) motion. Optionally a “click” sound is heard when one or more of piercing members 40, 42, 45, or 44 is being actuated.

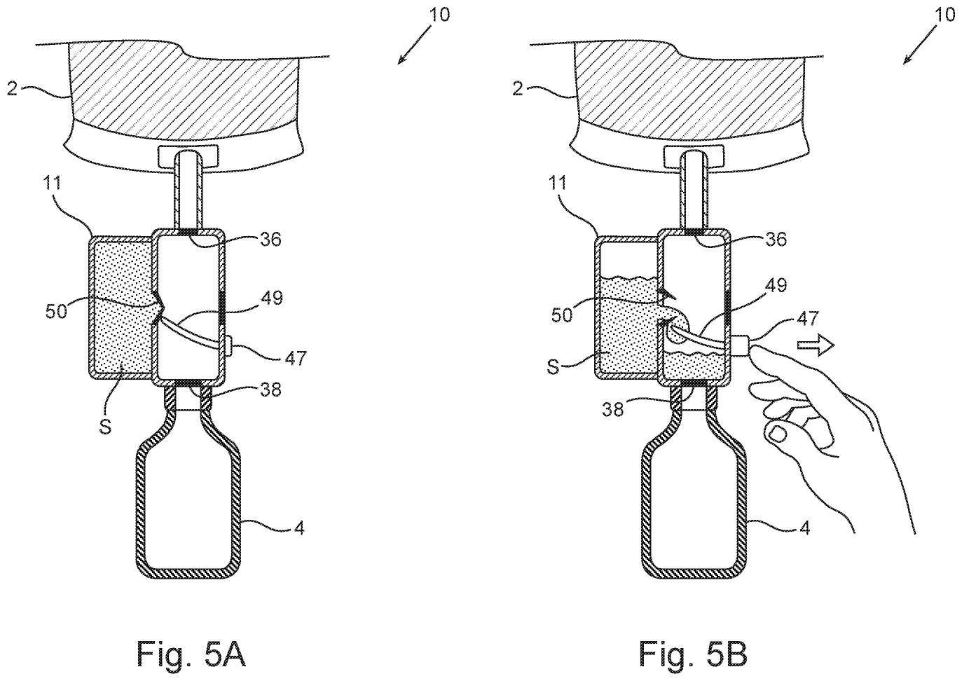

Referring now to FIGS. 4A-4C, a further alternative or additional embodiment of the herein disclosed device is shown. In this case, an entry valve 50 which may be a one-way valve or a two-way valve or any other suitable valve is provided to control opening and closing of entry aperture 28. Valve actuator 52 is adapted to allow opening of valve 50 via valve manipulator which in this case is an elongated bar 54 being connected to valve actuator 52 and pushes valve 50 to allow opening thereof when valve actuator 52 is pressed by a user. Sterilizing substance S may thereafter flow into second chamber 12 and decontaminate a surface of vessels 2 and 4. In this configuration valve actuator 52 extends from the external surface of first chamber 11 and bar 54 connected thereto and located within first chamber 11. In FIGS. 5A-5B an alternative mechanism of operating entry valve 50 is shown. In this case, a valve actuator 47 and valve manipulator 49 are shown. Here, the valve actuator 47 extends from the external surface of second chamber 12 and valve manipulator 49 disposed within second chamber 12 and holds entry valve 50 in a closed state. Upon pressing valve actuator 47, the bar/valve manipulator 49 is withdrawn, allowing opening of entry valve 50. A second valve which is configured to control opening and closing of exit aperture 30, such as exit valve 51 is further optionally contemplated (FIG. 6).

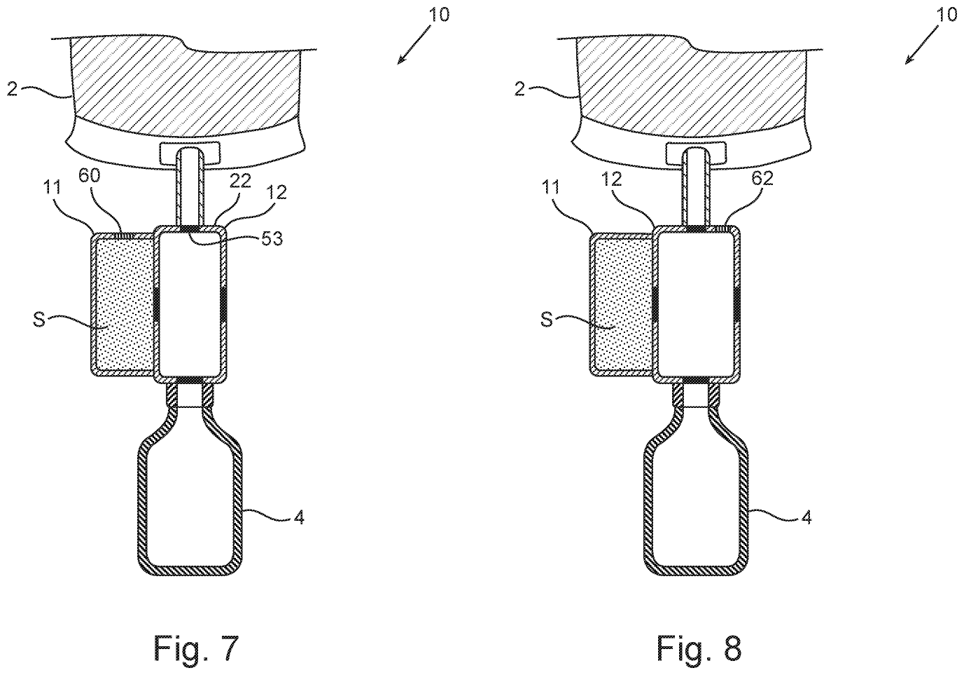

The herein disclosed device may optionally include one or more filters disposed in a wall, optionally, an aperture in first chamber 11 (see filter 60 in FIG. 7) and/or second chamber 12 (see filter 62 in FIG. 8) and provided to release pressure from the one or more chambers while preventing unfiltered air from entering the device. The filter is configured such to include pores restricting passage of the sterilizing substance S. The sterilizing substance S is thus maintained within the one or more first and second chambers 11 and 12, respectively. The filter may have pores having a diameter of about 5 microns, or less. In one or more embodiments, the filter may have pores having a diameter of 0.22 microns, or less.

FIGS. 9A-9D illustrate an alternative method and configuration for removing sterilizing substance S following decontamination of first and second vessel surfaces. In this case, a sponge 68 is provided within second chamber 12. Sponge 68 is configured to absorb sterilizing substance S following entry thereof within second chamber 12. Sponge 68 may be composed of any suitable sponge-like material, and/or highly absorptive material, expandable material, a combination thereof, or any of the alike. Optionally the sponge 68 is coupled to side wall 26 of second chamber 12 and sized and shaped such to allow absorption of the entire amount of sterilizing substance S that enters into second chamber 12. Alternatively, sponge 68 can be coupled to side wall 20. The sponge 68 is further configured such that even when saturated with sterilizing substance S and accordingly enlarged in size, a fluid passageway can be established between first vessel 2 and second vessel 4.

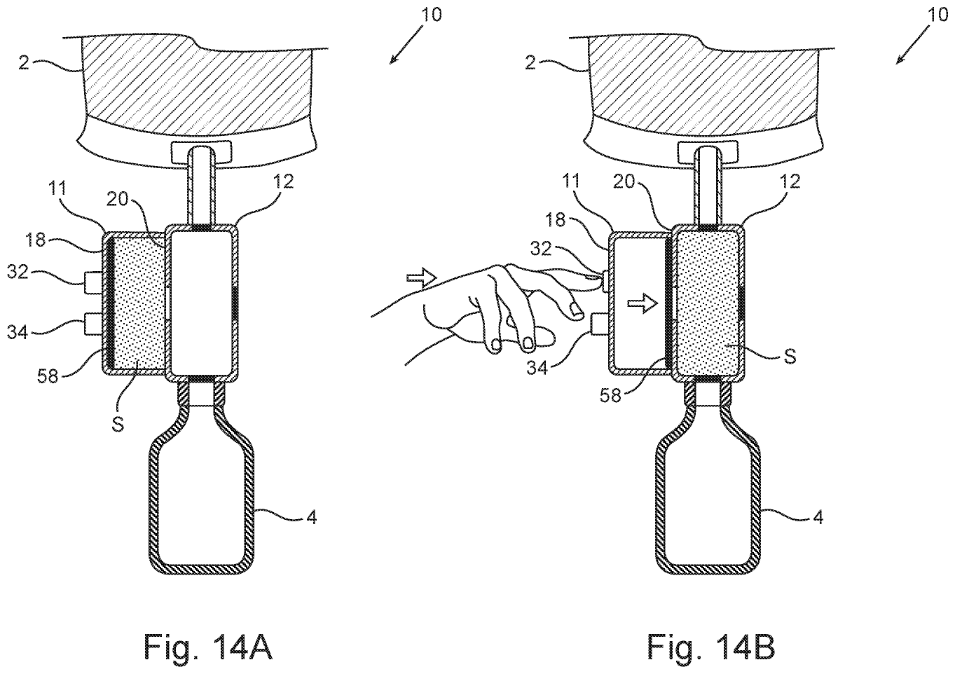

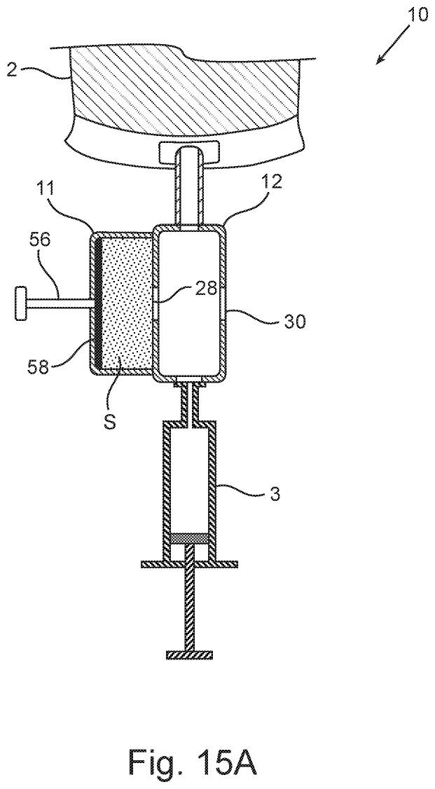

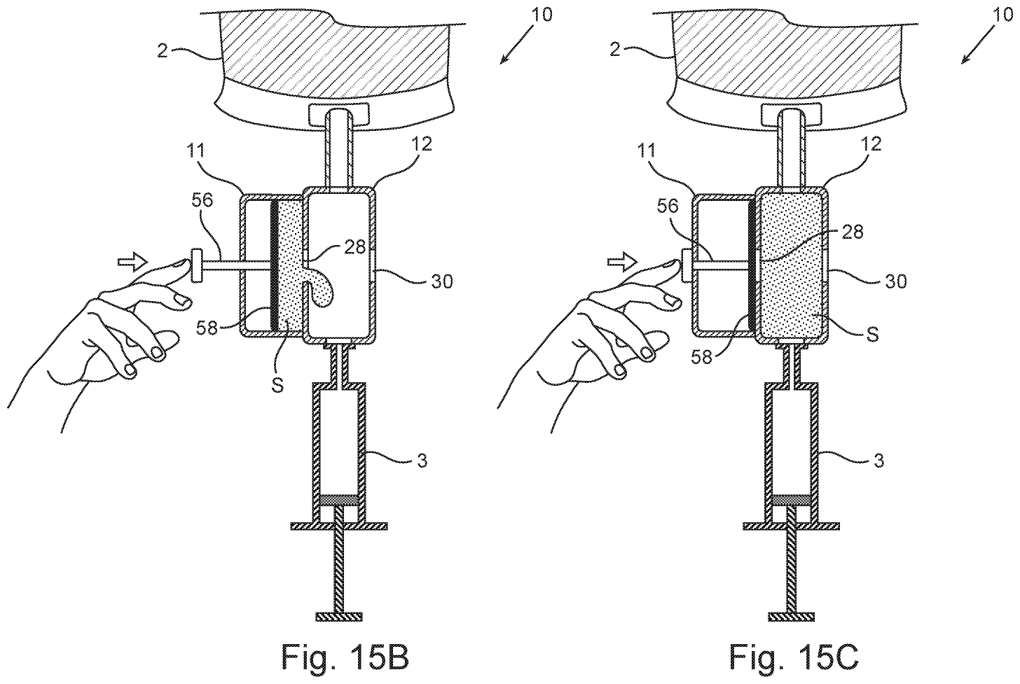

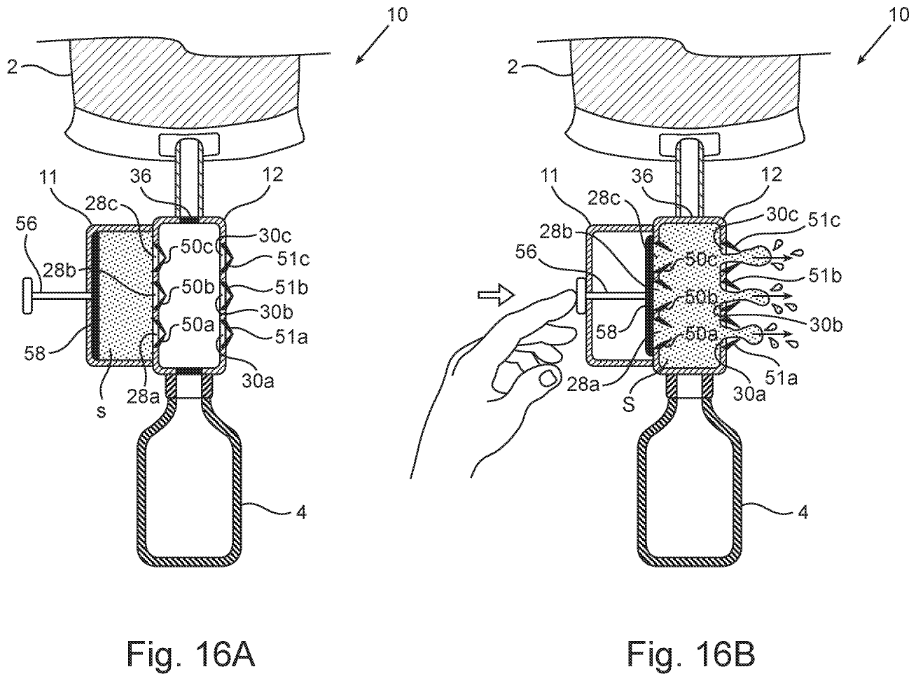

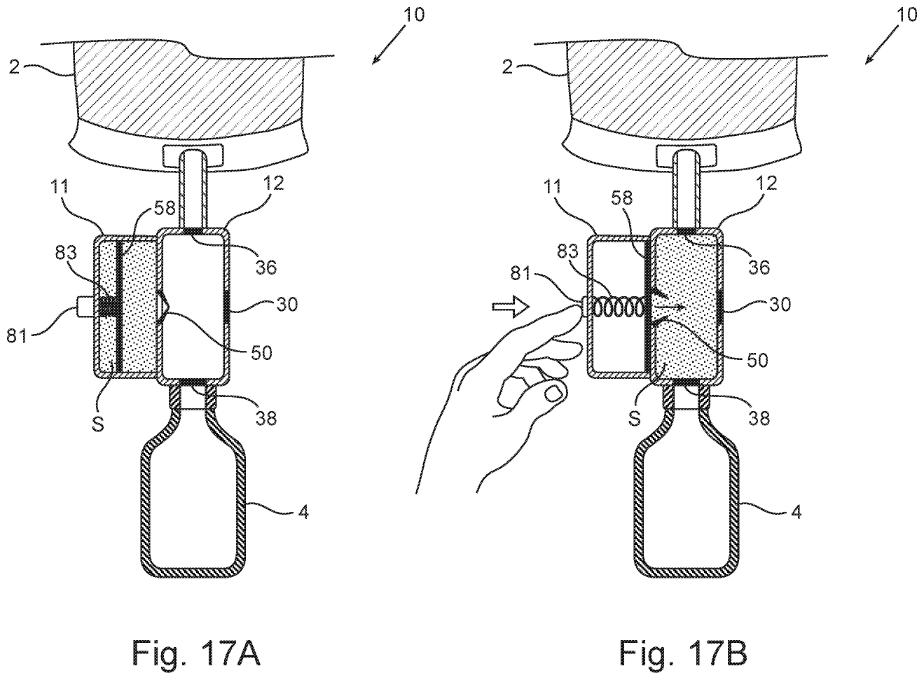

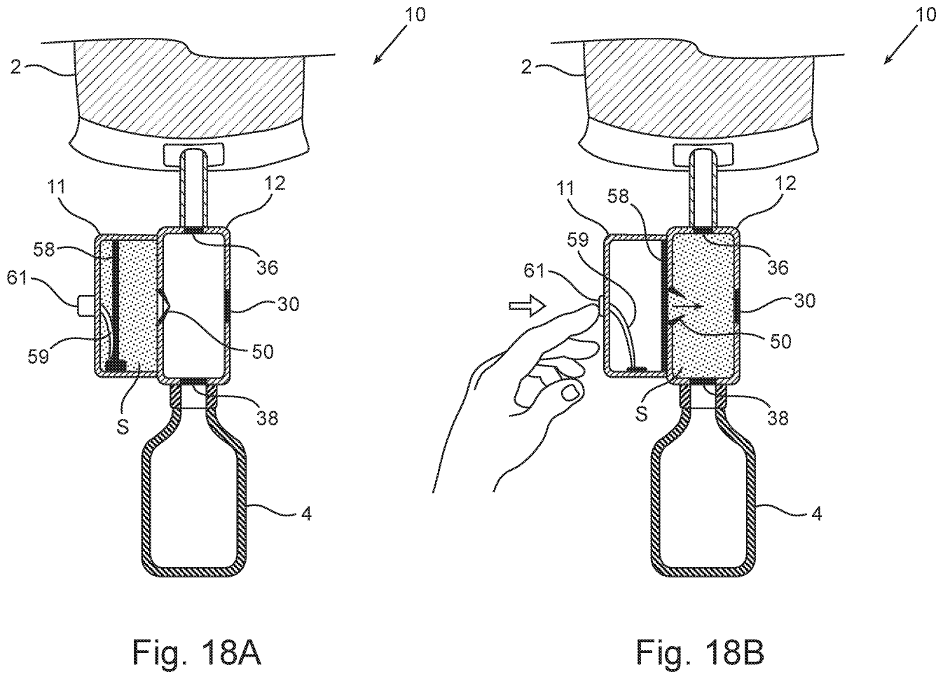

FIGS. 14A-14B depict an exemplary mechanism of transferring the sterilizing substance S from first chamber 11 into second chamber 12. A moveable shaft 58 located against side wall 18 can be used to push the sterilizing substance S that in this case may be a pressurized gas. Pressing actuator 32, unlocks the shaft 58 allowing movement thereof from left to right within chamber 11 and pressing sterilizing substance S towards second chamber 12. Optionally, movement of the shaft 58 is due to a high-pressure gas (not shown) between the actuator 32 and the shaft 58. Optionally, a spring 83 manipulated by spring actuator 81 is used to release moveable shaft 58 and allow movement thereof towards second chamber 12 (FIGS. 17A-17B). Alternatively, additionally or optionally, a safety mechanism 59 can be used to lock shaft 58, preventing premature movement towards second chamber 12 (FIGS. 18A-18B). Upon pressing safety mechanism actuator 61, the shaft 58 is released and travels along chamber 11, pushing sterilizing substance S into the second chamber 12. In FIGS. 15A-15 C, a handle 56 attached to shaft 58 can be used to push the sterilizing substance S from first chamber 11 to second chamber 12. FIGS. 16A-16B demonstrates a plurality of entry valves 50a, 50b, and 50c manipulating opening and closing of entry apertures 28a, 28b, and 28c, respectively, that are further optionally contemplated. Further, a plurality of exit valves 51a, 51b, and 51c manipulating opening and closing of exit apertures 30a, 30b, and 30c, respectively, are further optionally contemplated. Providing a plurality of entry aperture 28a, 28b, and 28c may provide for laminar and/or unidirectional flow of sterilizing substance S through chamber 12 thus more efficiently flushing chamber 12 of contaminants.

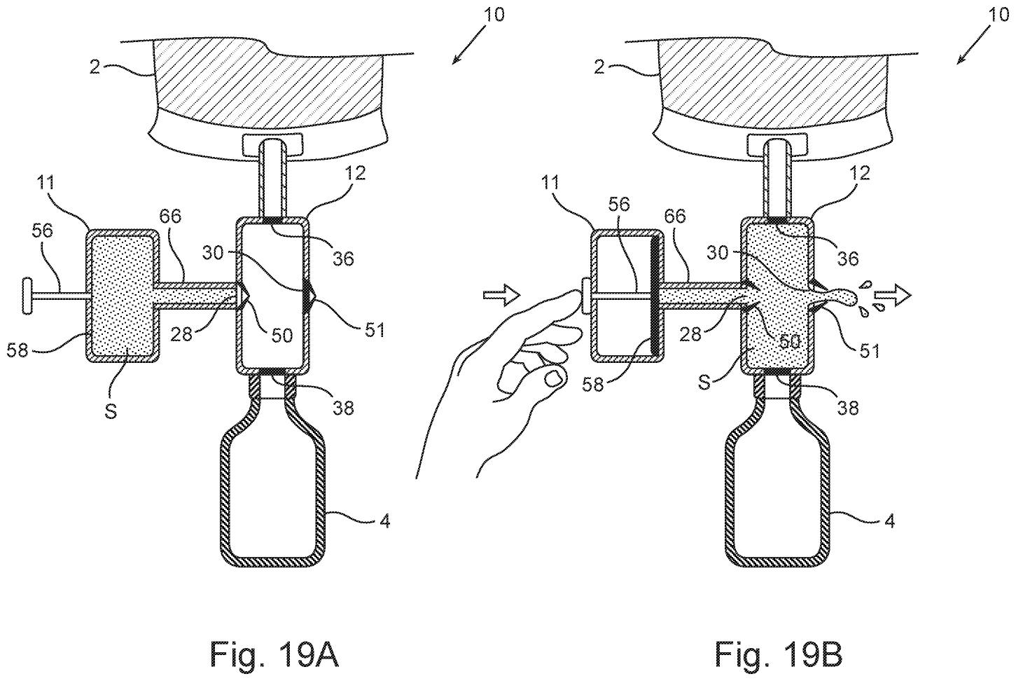

FIGS. 19A-19B illustrate yet a further optional embodiment of device 10 which includes entry channel 66 configured to provide for a laminar or unidirectional flow of sterilizing substance S. The laminar flow may allow for a high flow rate of the sterilizing substance S into the second chamber. The sterilizing substance S is maintained in first chamber 11 which is separated from second chamber 12, by entry conduit 66. Pushing handle 56, by a user, pushes sterilizing substance S from first chamber 11 towards second chamber 12 via entry conduit 66. The sterilizing substance S which enters into chamber 12 allows decontamination thereof. The sterilizing substance S is further pressed against exit valve 51, forcibly opening thereof, allowing exit of the sterilizing substance S from within the second chamber 12. In one or more embodiments, entry conduit 66 may have an inner diameter of less than 20 millimeters, less than 10 millimeters, less than 5 millimeters, less than 2.5 millimeters or less than 1 millimeters. The length of entry conduit 66 may be less than 1 inch, less than 0.5 inches, or less than 0.25 inches.

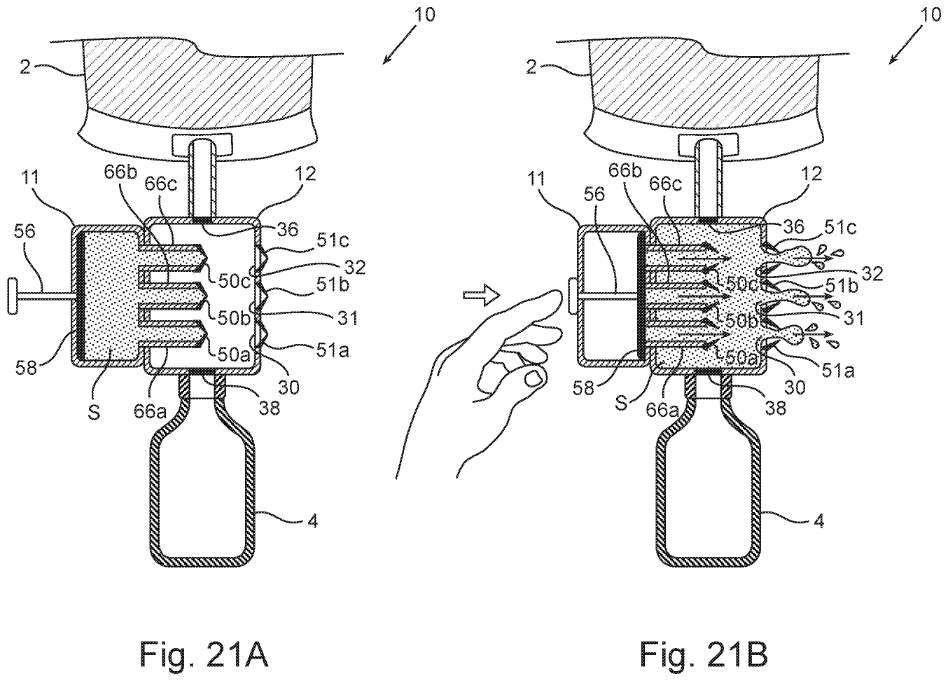

A plurality of entry channels is further optionally contemplated. For example, FIGS. 21A-21B illustrate device 10 which optionally includes three entry channels 66a, 66b and 66c which end or are enclosed by entry valves 50a, 50b and 50c, respectively. Pushing handle 56 towards second chamber 12 establishes a horizontal laminar or streamline flow of sterilizing substance S via entry channels 66a, 66b and 66c. Entry valves 50a, 50b and 50c are forced to open by the streamline and allow entrance to second chamber 12. The exit valves 51a, 51b and 51c are subsequently pushed by the current, allowing removal of the sterilizing substance S from the second chamber 12.

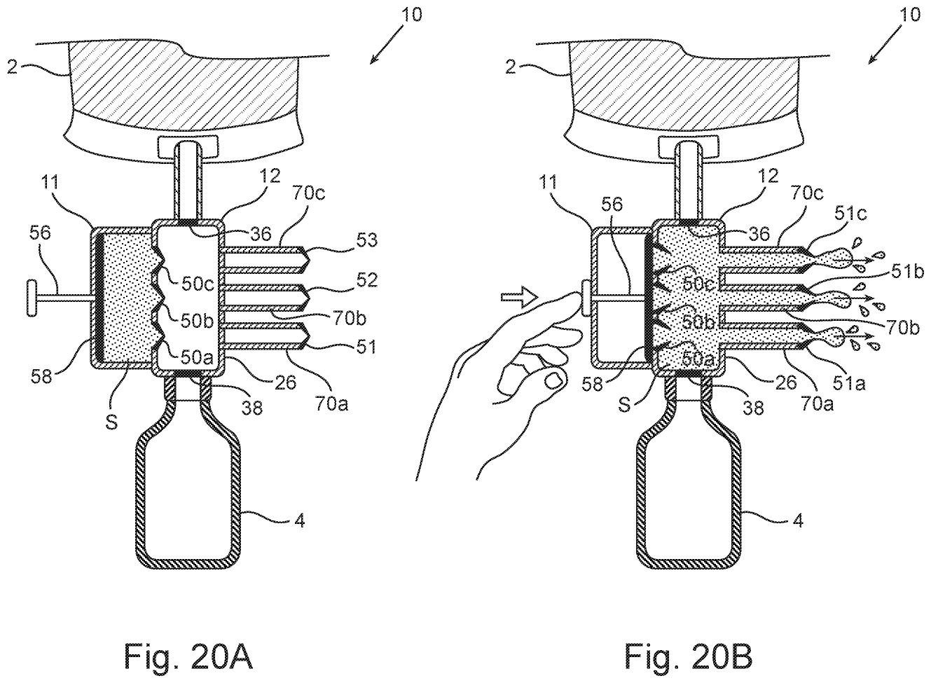

One or more exit channels are further contemplated and demonstrated in FIGS. 20A-20B. Here, the handle 56 attached to shaft 58 pushes sterilizing substance S into second chamber 12 via entry valves 50a, 50b, and 50c. Exit channels 70a, 70b and 70c enclosed by exit valves 51a, 51b, and 51c extend outwardly and horizontally from side wall 26 of second chamber 12. Sterilizing substance S that enters to second chamber 12 flows via exit channels 70a, 70b and 70c horizontally and/or laminarly and exit second chamber 12 via exit valves 51a, 51b and 51c.

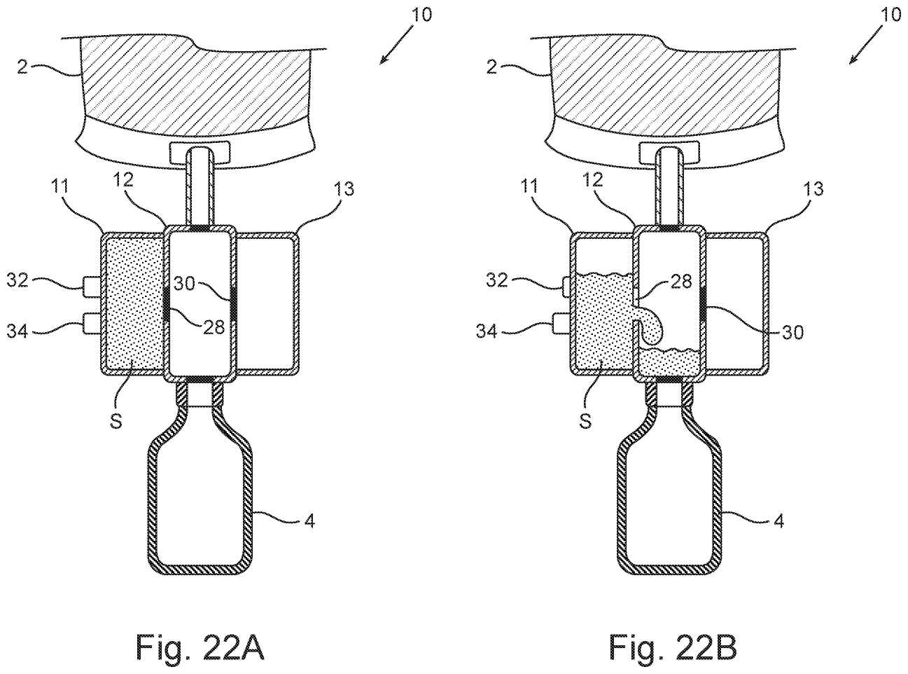

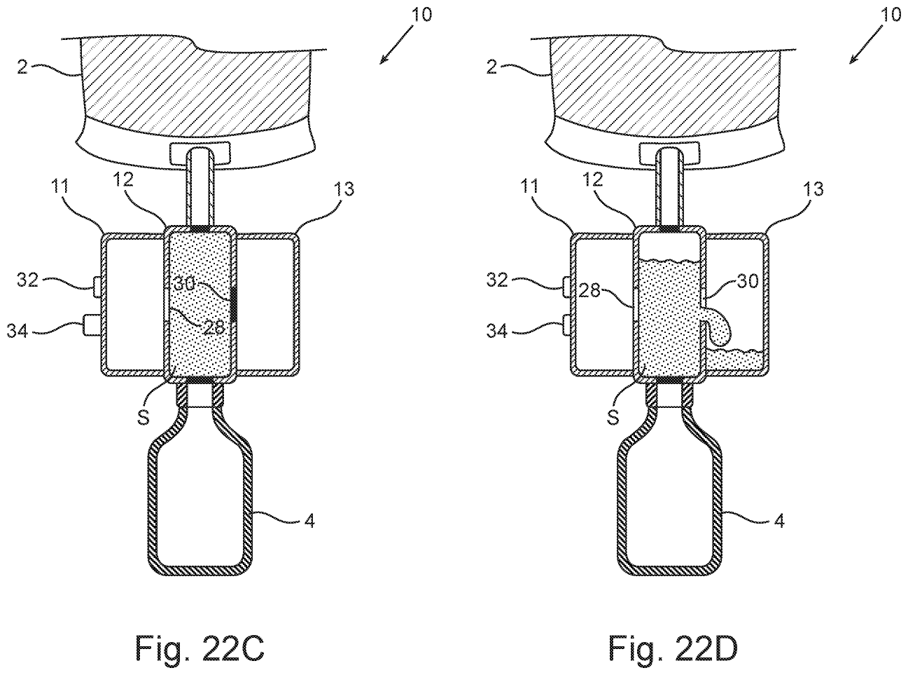

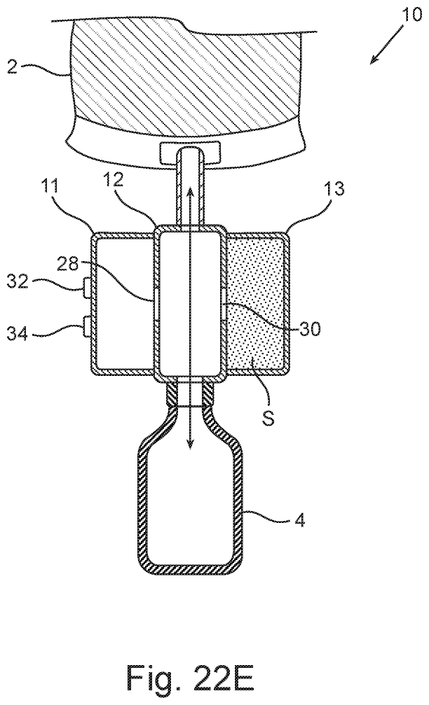

Reference is now made to FIGS. 22-26 demonstrating device 10 which may optionally include a third chamber 13. As demonstrated in FIGS. 22A-22E, third chamber 13 is provided next or parallel to second chamber 12 such that second chamber 12 is situated between first chamber 11 and third chamber 13. By adding a third chamber 13, the device 10 can be reusable. The sterilizing substance S after flushing second chamber 12 can be transferred into third chamber 13 and stored in said chamber 13 until further use is required.

Optionally, sterilizing substance S is being kept in first chamber 11 when in a highly pressurized state. Further optionally, or additionally, third chamber 13 has a negative pressure such that the sterilizing substance S may be sucked into third chamber 13 and fill thereof after entering second chamber 12. In One or more embodiments, first chamber 11 has a higher pressure than second chamber 12. In one or more embodiments, second chamber 12 has a lower pressure than first chamber 11. In one or more embodiments, second chamber 12 has a higher pressure than third chamber 13. In one or more embodiments, third chamber 13 has a lower pressure than second chamber 12.

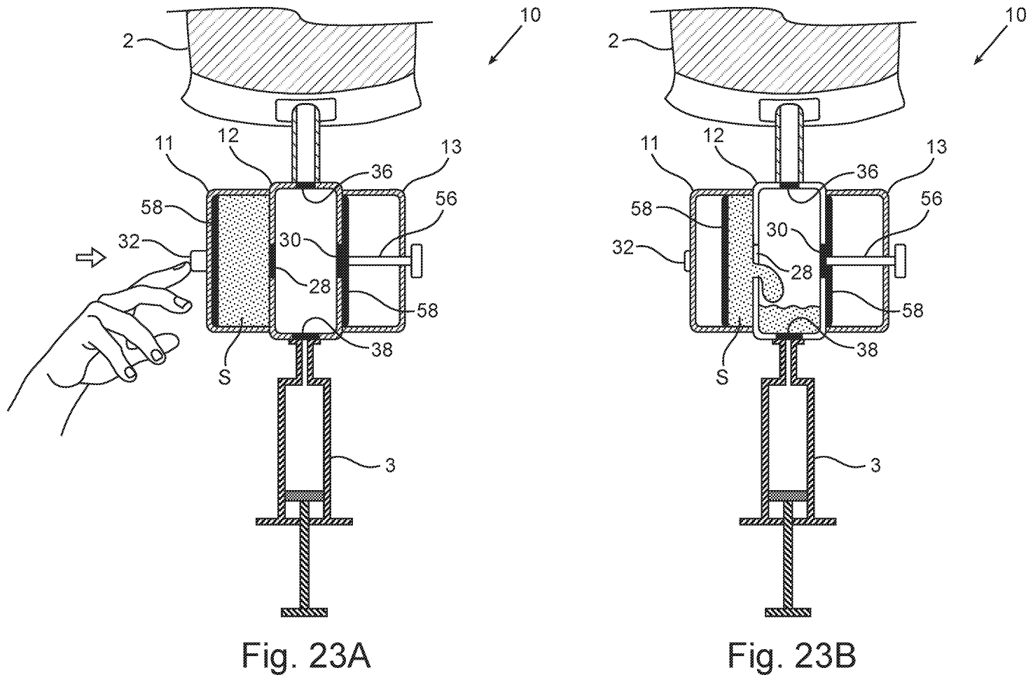

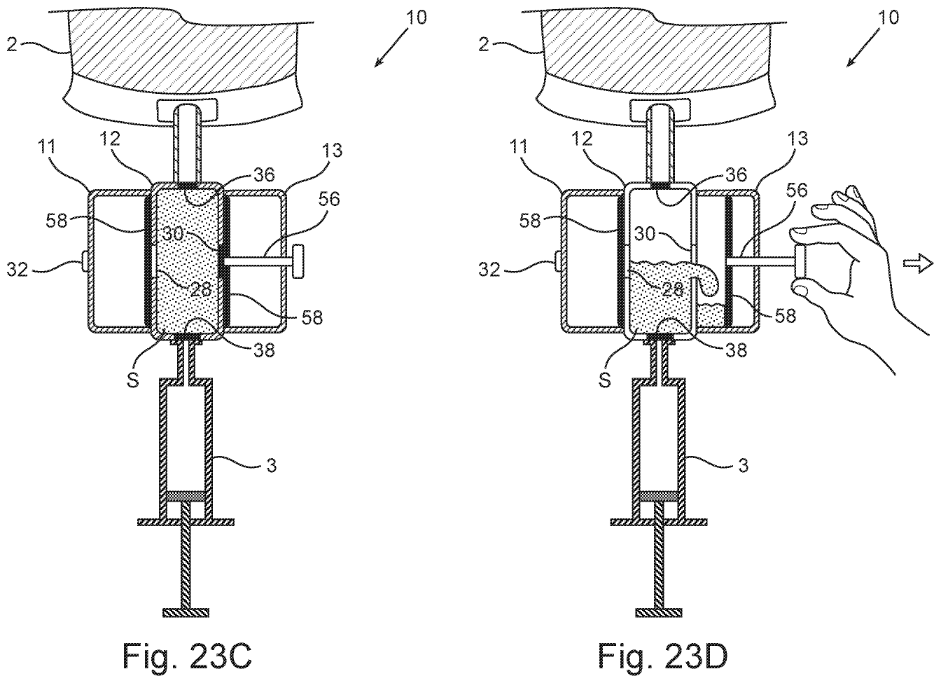

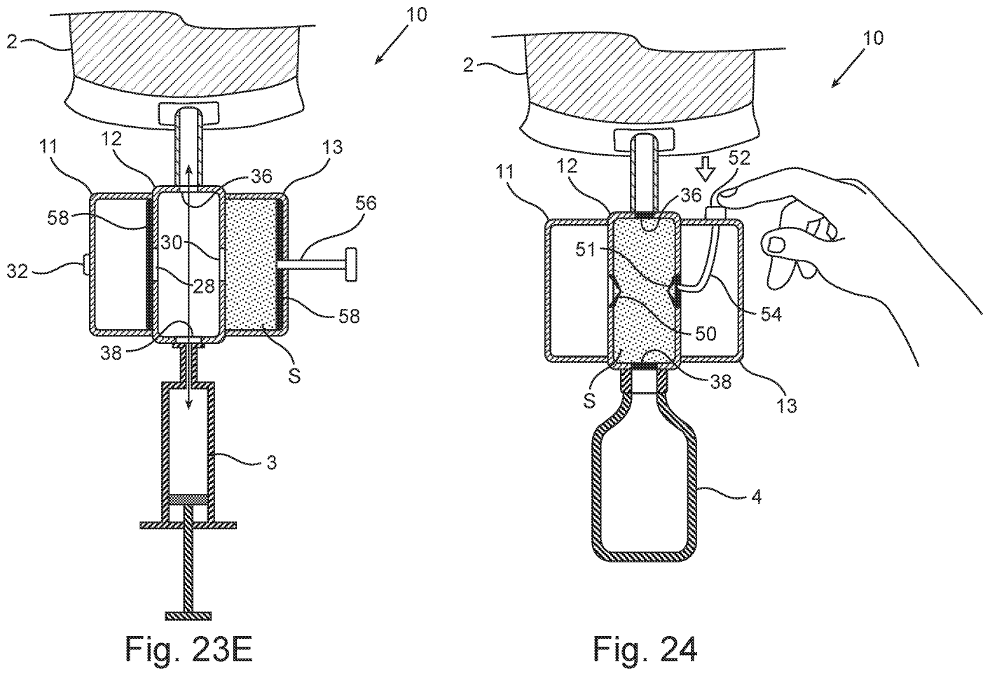

In FIGS. 23A-23E, a first transfer mechanism including shaft 58 is provided to push sterilizing substance S from first chamber 11 into second chamber 12 upon actuating actuator 32 (FIGS. 23A and 23C). A second transfer mechanism in the form of handle 56 and shaft 58 is further provided such that when handle 56 is pulled away (to the right) from chamber 13, a negative pressure (vacuum) is created drawing or sucking sterilizing substance S from second chamber 12 into third chamber 13 (FIGS. 23D and 23E).

FIG. 24 demonstrates valve actuator 52 and bar 54 located to control opening and closing of exit valve 51 from third chamber 13. Exit valve 51 is shown situated here in an opposite direction wherein opening thereof is performed towards second chamber 12.

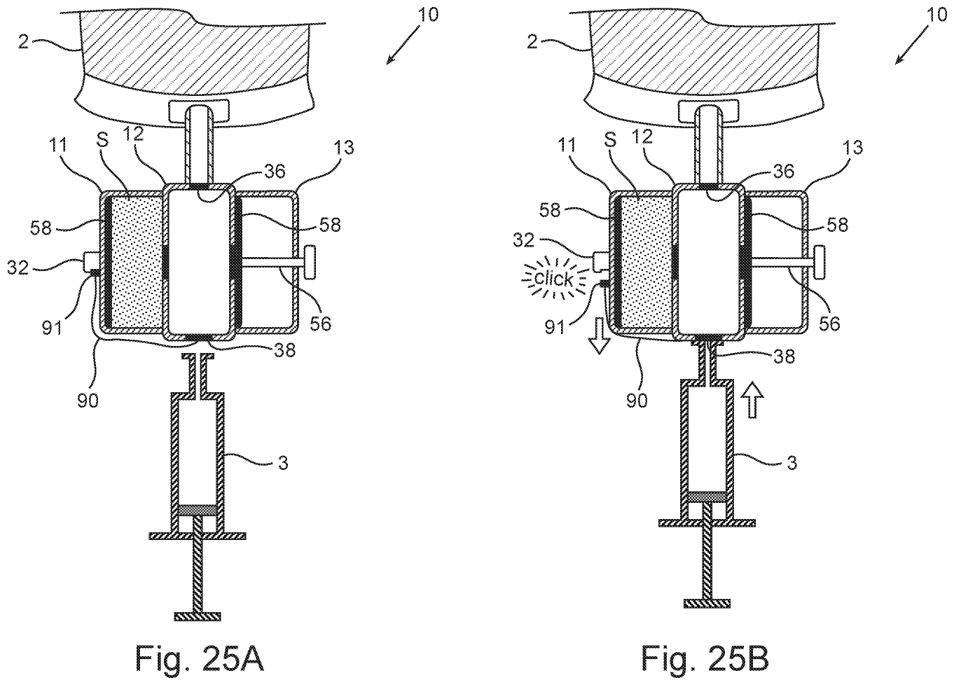

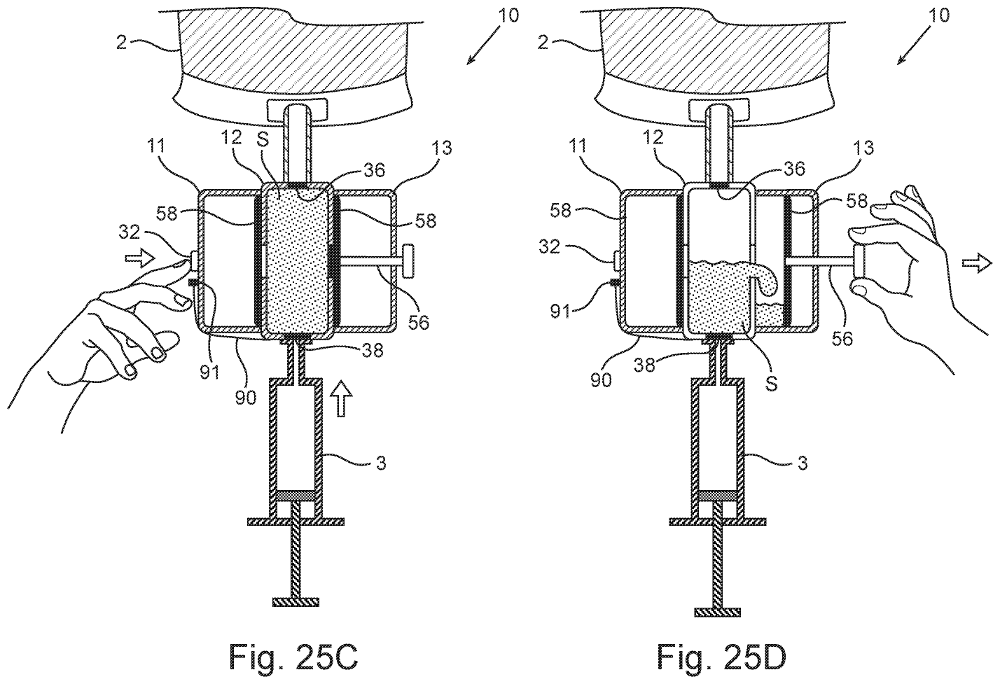

FIGS. 25A-25E depict a safety mechanism comprising pin 91 attached to cable 90 which can be used additionally or alternatively to the safety mechanism of FIGS. 18A-18B. In this safety mechanism, premature movement of shaft 58 is prevented by pin 91 being interlocked within actuator 32 (FIG. 25A). Cable 90 can release pin 91 from actuator 32 upon attachment of syringe 3 to connection port 38 (FIG. 25B). The cable 90 retracts pin 91 and the actuator 32 can then be activated by a user to thereby release shaft 58 and allow entrance of sterilizing substance S into second chamber 12 (FIG. 25C). Handle 56 can then be pulled by the user to transfer the sterilizing substance S to third chamber 13 (FIGS. 25D-25E). Other safety mechanisms that may prevent premature substance transfer and/or opening of the entry aperture, the entry actuator, the exit aperture, and/or the exit actuator are herein contemplated.

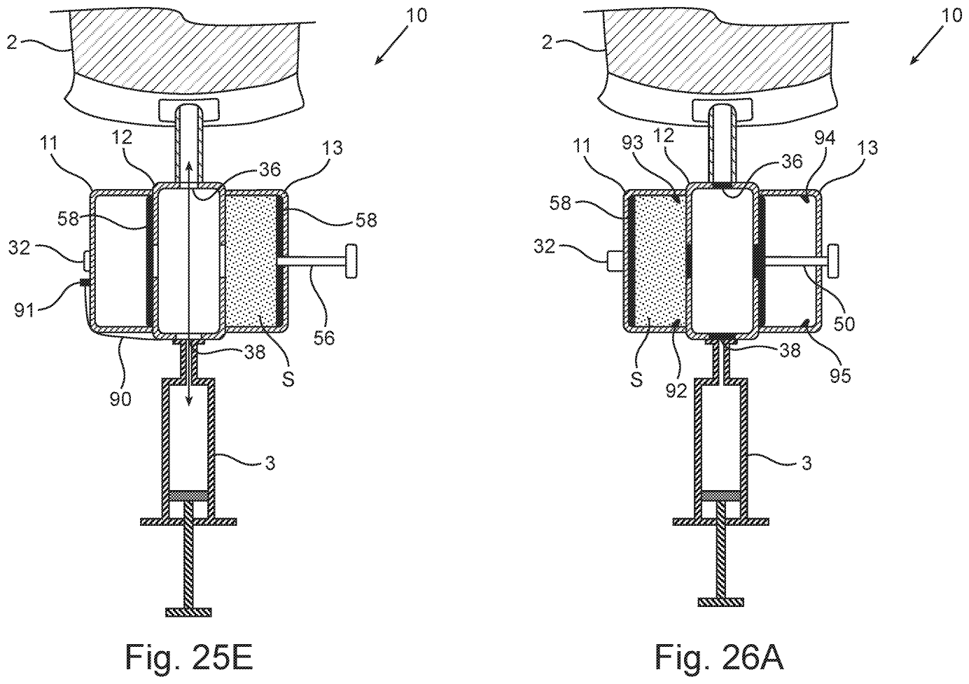

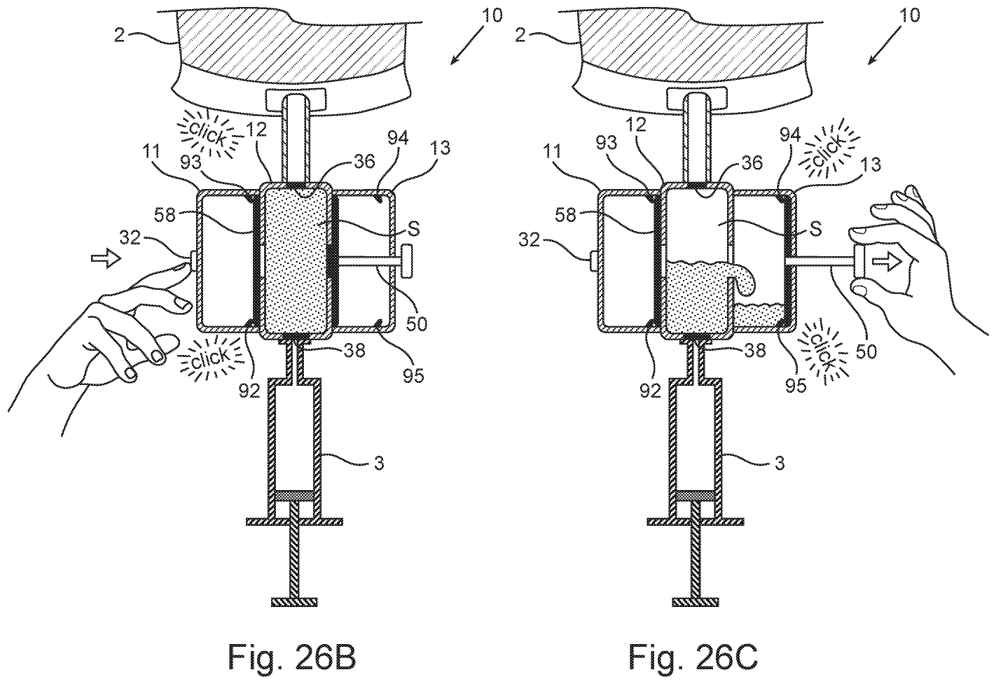

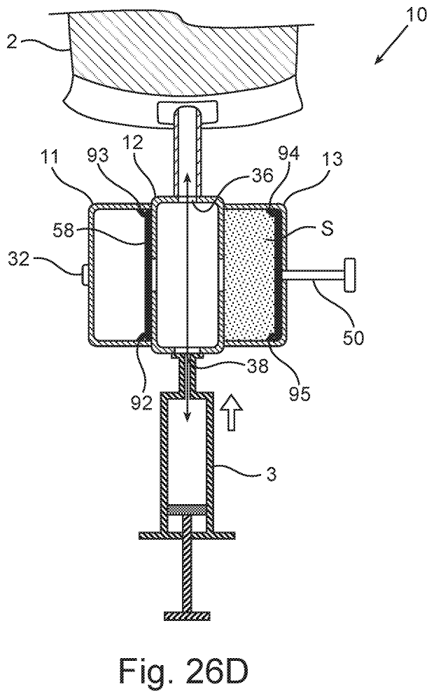

FIGS. 26A-26D depict a locking mechanism comprising brakes 92, 93, 94 and 95 configured to restrict movement of shaft 58 positioned both in chamber 11 and in chamber 13. The locking mechanism is therefore configured to prevent movement of shaft 58 from retracting back to the original position. Brakes 92 and 93 are positioned against bottom and top walls of first chamber 11 and brakes 95 and 94 positioned against a bottom wall and a top wall of chamber 13, respectively. Following movement of shaft 58 from left to the right, towards second chamber 12, the shaft 58 is locked in place against side wall 20. A “click” sound may be heard once shaft 58 being locked between brakes 92 and 93 (FIG. 26B). Similarly, shaft 58 which is disposed in third chamber 13, after being pulled by a user can be locked in place against side wall of chamber 13. A “click” sound may be heard once shaft 58 being locked between brakes 95 and 94 (FIG. 26C). A fluid passage way between vessels 2 and 3 may then be established. In one or more embodiments, brakes 92 and 93 may be configured to be pressed against an inner wall of chamber 11 when shaft 58 moves over or across brakes 92 and 93. Once shaft 58 reaches a final position adjacent to compartment 12 brakes 92 and 93 may be configured to retract back to their original position protruding out of the inner walls chamber 11 and thus locking shaft 58 in a final position. Similarly, in one or more embodiments, brakes, 94 and 95 may be configured to be pressed against an inner wall of chamber 13 when shaft 58 moves over or across brakes 94 and 95. Once shaft 58 reaches a final position pressed against the right side wall of chamber 13 brakes 94 and 95 may be configured to retract back to their original position protruding out of the inner walls of chamber 13 and thus locking shaft 58 in a final position within chamber 13.

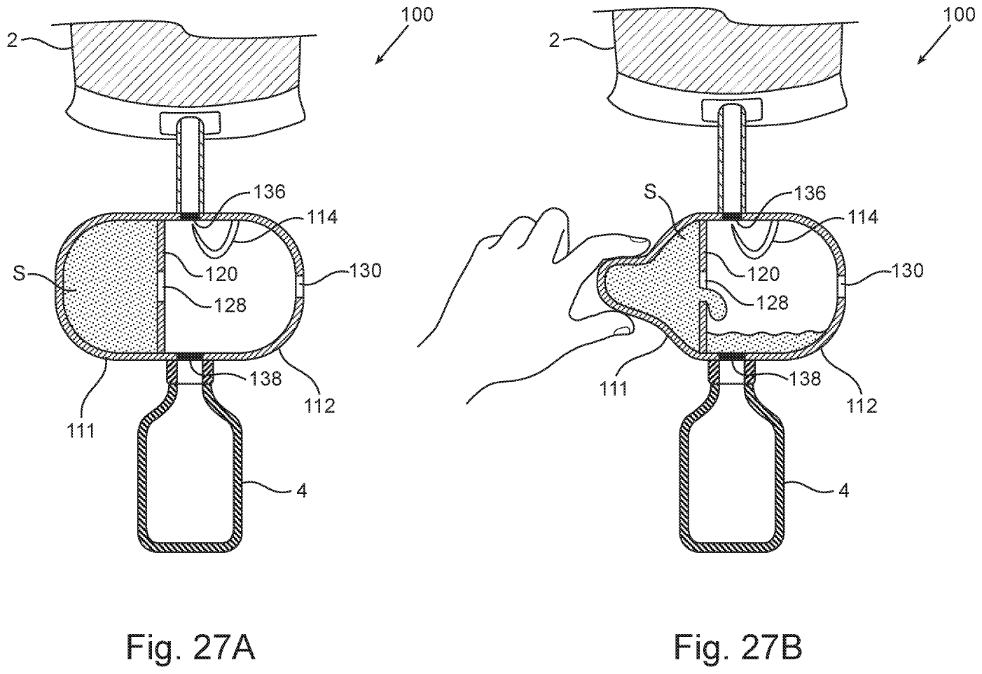

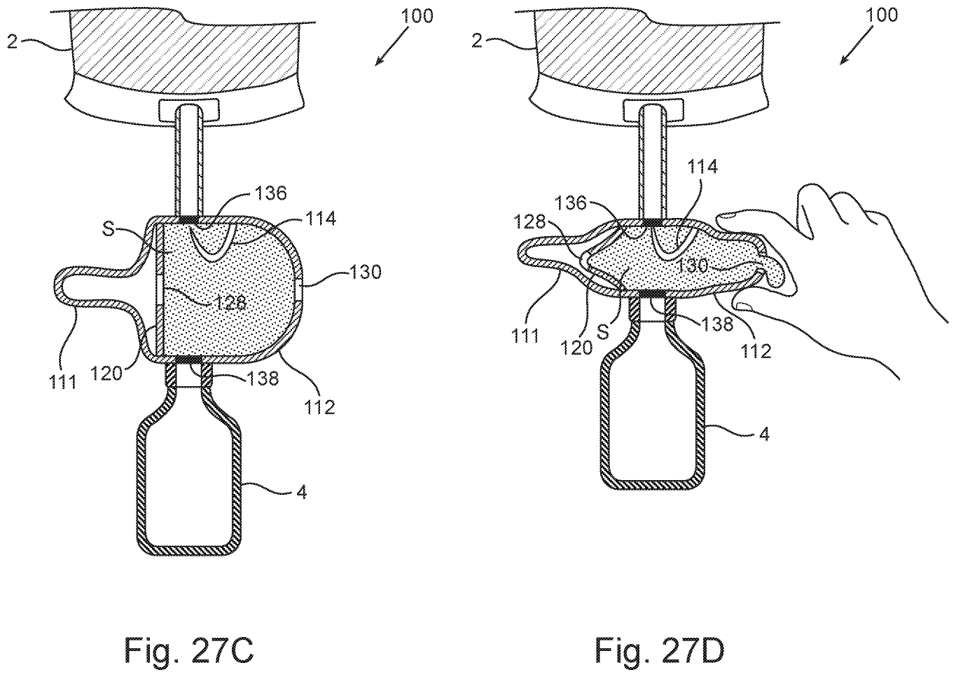

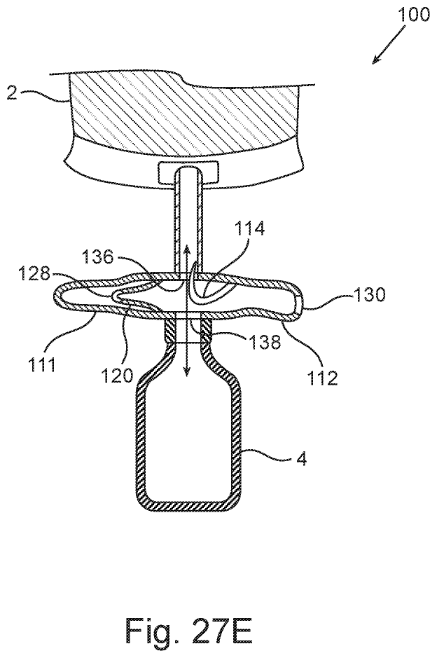

Referring now to FIGS. 27A-27E, yet another exemplary device 100 as herein disclosed is illustrated. Device 100 is similar to device 10 with the exception that it is collapsible, or flexible. Namely, the device 100 is surrounded by a wall, optionally rounded wall which is collapsible, and optionally manufactured from a resilient material. Device 100 includes a first chamber 111 and a second chamber 112 which share a side wall 120. Sterilizing substance S is housed in the first chamber 111. Movement of sterilizing substance S is afforded via manually pressing or collapsing first chamber 111 thereby forcibly transferring said substance S into second chamber 112 via entry aperture 128 (FIGS. 27B and 27C). By manually pressing or squeezing second chamber 112 the sterilizing chamber S can be removed from second chamber 112 via exit aperture 130 (FIG. 27D). One or more piercing members such as piercing member 114 can be provided to allow opening of first connection port 136, optionally by pressing from the outside piercing member 114. Similarly, one or more piercing members 114 may be provided to allow opening of second connection port 138 and/or of exit aperture 130. When connection ports 136 and 138 are opened a fluid passageway may be formed between first and second vessels 2 and 4, respectively (FIG. 27E). The device 100 is optionally provided with one or more covers and/or valves and/or filters, etc., as disclosed herein for device 10.

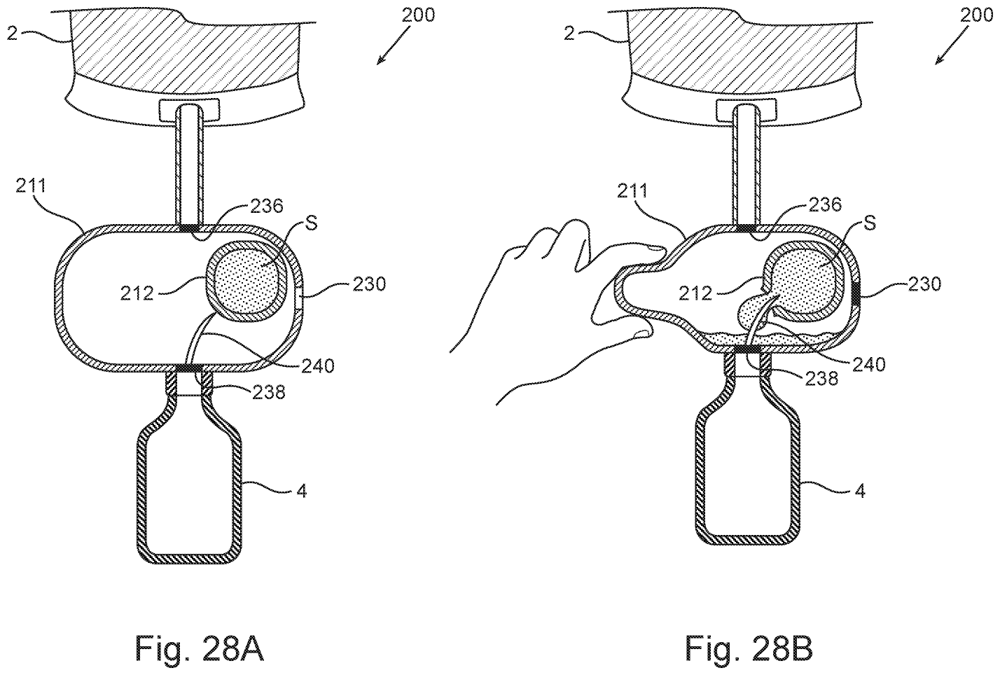

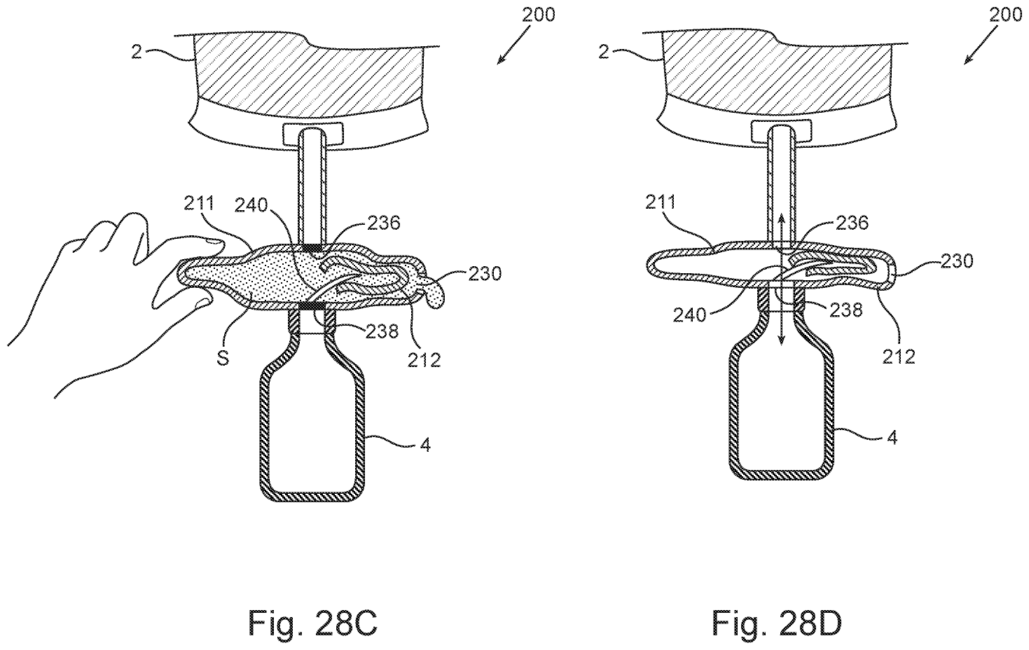

Referring now to FIGS. 28A-28D, yet another exemplary collapsible device 200 is illustrated. Device 200 includes a first chamber 211 and a second chamber 212, wherein the second chamber 212 is disposed within first chamber 211. Upon manually pressing or collapsing first chamber 211, a piercing member 240 provided within first chamber 211 is being capable of puncturing second chamber 212 (FIG. 28B). The sterilizing substance S is spilled into first chamber 211 and decontaminates the surfaces of vessels 2 and 4. Further or continuous pressing first chamber 211 creates a pressure that presses sterilizing substance S out of first chamber 211 via exit aperture 230 (FIG. 28C). A fluid passageway can then be formed between first vessel 2 and second vessel 4. Optionally, device 200 may not have a piercing member 240. In this embodiments, upon manually pressing or collapsing first chamber 211 second chamber 212 is also collapsed and ruptures thus spilling or releasing sterilizing substance S in first chamber 211 thus decontaminating a surface of vessel 2 and 4.

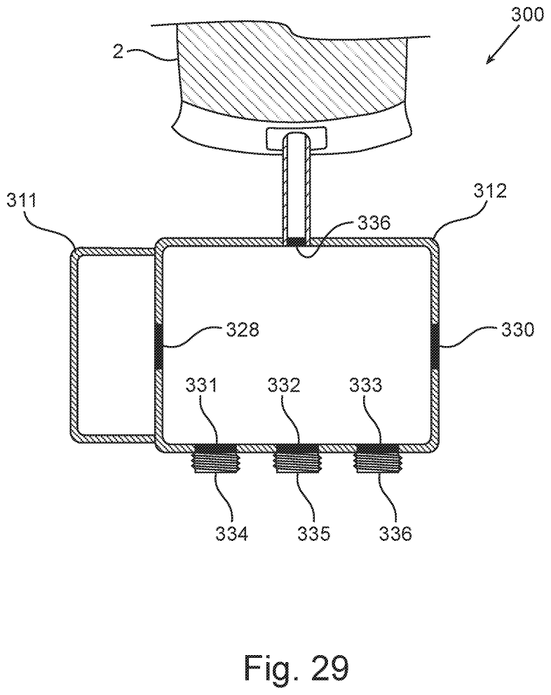

FIG. 29 illustrates yet a further exemplary device 300 that facilities decontamination of and engagement between a plurality of vessels. Similarly, to device 10, device 300 includes a first chamber 311 and a second chamber 312 with an entry aperture 328 and an exit aperture 330. The device 300 allows an engagement between the second chamber 312 and a plurality of vessels. Three connection ports 331, 332 and 333 are shown attached to threads 334, 335 and 336, respectively, but two, or more than three ports and vessel connections are contemplated.

Each of the following terms: ‘includes’, ‘including’, ‘has’, ‘having’, ‘comprises’, and ‘comprising’, and, their linguistic, as used herein, means ‘including, but not limited to’, and is to be taken as specifying the stated component(s), feature(s), characteristic(s), parameter(s), integer(s), or step(s), and does not preclude addition of one or more additional component(s), feature(s), characteristic(s), parameter(s), integer(s), step(s), or groups thereof. Each of these terms is considered equivalent in meaning to the phrase ‘consisting essentially of’.

Each of the phrases ‘consisting of’ and ‘consists of’, as used herein, means ‘including and limited to’.

The term ‘method’, as used herein, refers to steps, procedures, manners, means, or/and techniques, for accomplishing a given task including, but not limited to, those steps, procedures, manners, means, or/and techniques, either known to, or readily developed from known steps, procedures, manners, means, or/and techniques, by practitioners in the relevant field(s) of the disclosed invention.

Throughout this disclosure, a numerical value of a parameter, feature, characteristic, object, or dimension, may be stated or described in terms of a numerical range format. Such a numerical range format, as used herein, illustrates implementation of some exemplary embodiments of the invention, and does not inflexibly limit the scope of the exemplary embodiments of the invention. Accordingly, a stated or described numerical range also refers to, and encompasses, all possible sub-ranges and individual numerical values (where a numerical value may be expressed as a whole, integral, or fractional number) within that stated or described numerical range. For example, a stated or described numerical range ‘from 1 to 6’ also refers to, and encompasses, all possible sub-ranges, such as ‘from 1 to 3’, ‘from 1 to 4’, ‘from 1 to 5’, ‘from 2 to 4’, ‘from 2 to 6’, ‘from 3 to 6’, etc., and individual numerical values, such as ‘1’, ‘1.3’, ‘2’, ‘2.8’, ‘3’, ‘3.5’, ‘4’, ‘4.6’, ‘5’, ‘5.2’, and ‘6’, within the stated or described numerical range of ‘from 1 to 6’. This applies regardless of the numerical breadth, extent, or size, of the stated or described numerical range.

Moreover, for stating or describing a numerical range, the phrase ‘in a range of between about a first numerical value and about a second numerical value’, is considered equivalent to, and meaning the same as, the phrase ‘in a range of from about a first numerical value to about a second numerical value’, and, thus, the two equivalently meaning phrases may be used interchangeably.

The term ‘about’, is some embodiments, refers to ±30% of the stated numerical value. In further embodiments, the term refers to ±20% of the stated numerical value. In yet further embodiments, the term refers to ±10% of the stated numerical value.

It is to be fully understood that certain aspects, characteristics, and features, of the invention, which are, for clarity, illustratively described and presented in the context or format of a plurality of separate embodiments, may also be illustratively described and presented in any suitable combination or sub-combination in the context or format of a single embodiment. Conversely, various aspects, characteristics, and features, of the invention which are illustratively described and presented in combination or sub combination in the context or format of a single embodiment, may also be illustratively described and presented in the context or format of a plurality of separate embodiments.

Although the invention has been described in conjunction with specific embodiments thereof, it is evident that many alternatives, modifications, and variations will be apparent to those skilled in the art. Accordingly, it is intended to embrace all such alternatives, modifications, and variations that fall within the spirit and broad scope of the appended claims.

All publications, patents, and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A device for decontaminating a surface of at least one vessel, the device comprising:
a housing comprising
a first chamber configured to accommodate a sterilizing/disinfecting substance; and
a second chamber, connected to the first chamber and configured to connect the at least one vessel, the second chamber further configured to receive the sterilizing substance from the first chamber and to thereby decontaminate the surface of the at least one vessel.

2. The device of claim 1, wherein an entry aperture is disposed between the first chamber and the second chamber, the entry aperture is configured to allow the sterilizing/disinfecting substance to enter from the first chamber to the second chamber.

3. The device of claim 2, wherein the entry aperture is sealed by a cover, or wherein the entry aperture includes a one-way valve.

4. The device of claim 1, wherein the second chamber comprises an exit aperture for allowing removal of the sterilizing/disinfecting substance from the second chamber.

5. The device of claim 4, wherein the exit aperture is sealed by a cover, or wherein the exit aperture includes a one-way valve.

6. The device of claim 2, further comprising an entry actuator operable to manipulate opening of the entry aperture.

7. The device of claim 6, wherein opening the entry aperture includes opening a valve or a cover disposed along the entry aperture.

8. The device of claim 6, wherein the entry actuator is connected to the first chamber from an exterior surface thereof.

9. The device of claim 1, wherein the second chamber comprises a first connection port configured for coupling to a first vessel, wherein the first connection port provides a connection between the second chamber and the first vessel.

10. The device of claim 9, further comprising a second connection port configured for coupling to a second vessel, wherein the second connection port provides a connection between the second chamber and the second vessel.

11. The device of claim 1, wherein the sterilizing substance is selected from the group consisting of an antibacterial substance, an antiviral substance, an anti-tuberculin substance, and a combination thereof.

12. The device of claim 1, wherein the first chamber has a greater pressure than the second chamber.

13. The device of claim 1, further comprising a sterilizing substance transfer mechanism configured to allow transferring of the sterilizing substance from the first chamber to the second chamber.

14. The device of claim 1, wherein the second chamber has one or more engagement mechanisms to connect the at least one vessel to the second chamber.

15. The device of claim 1, further comprising a one or more piercing members configured to pierce through a surface of the at least one vessel.

16. The device of claim 1, further comprising one or more horizontal channels between the first chamber and the second chamber, the one or more horizontal channels allowing a laminar flow of the sterilizing/disinfecting substance.

17. The device of claim 1, further comprising a fluid transfer mechanism allowing movement of the sterilizing/disinfecting substance between the first and the second chambers.

18. The device of claim 1, further comprising a safety mechanism configured to prevent premature entry of the sterilizing/disinfecting substance into the second chamber.

19. The device of claim 1, further comprising a third chamber configured to receive the sterilizing/disinfecting substance from the second chamber.

20. A method of decontaminating a surface of at least one vessel, the method comprising:
providing a decontamination device comprising a first chamber configured to accommodate a sterilizing/disinfecting substance and a second chamber, connected to the first chamber and configured to receive the sterilizing/disinfecting substance from the first chamber;
providing at least one vessel having a surface;
connecting the at least one vessel to the second chamber; and
transferring the sterilizing/disinfecting substance from the first chamber to the second chamber, thereby decontaminating the surface of the at least one vessel.
